SEQUENCE LISTING (35 sequences)

SEQ ID NO: 1 - protein, 7 aa, synthetic construct
PKKKRKV

SEQ ID NO: 2 - protein, 8 aa, synthetic construct
PAAKRVKL

SEQ ID NO: 3 - protein, 9 aa, synthetic construct
PAAKRVKLD

SEQ ID NO: 4 - protein, 7 aa, synthetic construct
VKRKKKP

SEQ ID NO: 5 - protein, 4 aa, synthetic construct
GGGS

SEQ ID NO: 6 - protein, 4 aa, synthetic construct
KSGG

SEQ ID NO: 7 - protein, 5 aa, synthetic construct
CGGGS

SEQ ID NO: 8 - protein, 4 aa, synthetic construct
CGGS

SEQ ID NO: 9 - protein, 30 aa, synthetic construct
WEARLARALARALARHLARALARALRACEA

SEQ ID NO: 10 - protein, 37 aa, synthetic construct
WEARLARALARALARHLARALARALRACEAPKKKRKV

SEQ ID NO: 11 - protein, 21 aa, synthetic construct
KARKKKLNKKGRKMAGRKRGR

SEQ ID NO: 12 - protein, 21 aa, synthetic construct
  VARIANT 5: note = any amino acid
  VARIANT 8: note = any amino acid
  VARIANT 9: note = any amino acid
  VARIANT 13: note = any amino acid
KARKXKLXXKGRXMAGRKRGR

SEQ ID NO: 13 - protein, 21 aa, synthetic construct
KARKAKLRLKGRLMAGRKRGR

SEQ ID NO: 14 - protein, 23 aa, synthetic construct
KARKKKLNKKGRKMAGRKRGRPK

SEQ ID NO: 15 - protein, 23 aa, synthetic construct
KARKAKLRLKGRLMAGRKRGRPK

SEQ ID NO: 16 - protein, 22 aa, synthetic construct
KARKAKLRLKGRLMAGRKRGRP

SEQ ID NO: 17 - protein, 22 aa, synthetic construct
KARKKKLNKKGRKMAGRKRGRP

SEQ ID NO: 18 - protein, 24 aa, synthetic construct
KARKAKLRLKARLWARHRARACEA

SEQ ID NO: 19 - protein, 22 aa, synthetic construct
KARKAKLRLKGRLWARHRACEA

SEQ ID NO: 20 - protein, 28 aa, synthetic construct
KARKKKLNKKGRKMAGRKRGRPKKKRKV

SEQ ID NO: 21 - protein, 36 aa, synthetic construct
KARKKKLNKKGRKMAGRKRGRPKGGGSPAAKRVKLD

SEQ ID NO: 22 - protein, 34 aa, synthetic construct
KARKAKLRLKGRLMAGRKRGRPKKSGGVKRKKKP

SEQ ID NO: 23 - protein, 36 aa, synthetic construct
KARKAKLRLKGRLMAGRKRGRPKGGGSPAAKRVKLD

SEQ ID NO: 24 - protein, 35 aa, synthetic construct
KARKAKLRLKGRLMAGRKRGRPCGGGSPAAKRVKL

SEQ ID NO: 25 - protein, 34 aa, synthetic construct
KARKAKLRLKGRLMAGRKRGRPCGGSPAAKRVKL

SEQ ID NO: 26 - protein, 33 aa, synthetic construct
KARKKKLNKKGRKMAGRKRGRPCGGSPKKKRKV

SEQ ID NO: 27 - protein, 33 aa, synthetic construct
KARKAKLRLKGRLMAGRKRGRPCGGSPKKKRKV

SEQ ID NO: 28 - protein, 32 aa, synthetic construct
KARKAKLRLKARLWARHRARACEAPAAKRVKL

SEQ ID NO: 29 - protein, 30 aa, synthetic construct
KARKAKLRLKGRLWARHRACEAPAAKRVKL

SEQ ID NO: 30 - protein, 29 aa, synthetic construct
KARKAKLRLKGRLWARHRACEAPKKKRKV

SEQ ID NO: 31 - protein, 30 aa, synthetic construct
WEAKLAKALAKALAKHLAKALAKALKACEA

SEQ ID NO: 32 - protein, 37 aa, synthetic construct
WEAKLAKALAKALAKHLAKALAKALKACEAPKKKRKV

SEQ ID NO: 33 - skipped

SEQ ID NO: 34 - protein, 7 aa, synthetic construct
GGSGGGS

SEQ ID NO: 35 - protein, 11 aa, synthetic construct
GGSGGGSGGGS

(19) United States
(12) Patent Application Publication
(10) Pub. No.: US 2025/0325699 A1
(43) Pub. Date: Oct. 23, 2025

(54) POLYPEPTIDES AND METHODS OF USE

(71) Applicant: Aldevron, LLC, Fargo, ND (US)

(72) Inventors: Amit Singh, Tewksbury, MA (US); Jodi Kennedy, Dedham, MA (US)

(21) Appl. No.: 18/856,479

(22) PCT Filed: Apr. 14, 2023
(86) PCT No.: PCT/US2023/065763

§ 371 Date: Oct. 11, 2024

Related U.S. Application Data
(60) Provisional application No. 63/331,452, filed on Apr. 15, 2022.

(51) Int. Cl. A61K 48/00; A61K 9/1272; A61K 9/51; C07K 14/47; C12N 15/88; G01N 33/68

(52) U.S. Cl. CPC A61K 48/005; A61K 9/1272; A61K 9/5123; C07K 14/47; C12N 15/88; G01N 33/68; C07K 2319/09; C07K 2319/80

(57) ABSTRACT

The invention features polypeptides containing nuclear localization sequences that can be used to deliver polynucleotides to a cell. The polypeptides can be formulated with a polynucleotide and a lipid.

### SEQUENCE LISTING

The instant application contains a Sequence Listing which has been submitted electronically in XML format and is hereby incorporated by reference in its entirety. Said XML copy, created on Apr. 10, 2023, is named 51503-077WO2_Sequence_Listing_4_10_23.XML and is 32,584 bytes in size.

### FIELD OF THE INVENTION

In general, the invention relates to polypeptides useful for delivery of therapeutics such as polynucleotides.

### BACKGROUND

Gene therapy is emerging as a promising approach to treat a wide variety of diseases and disorders in human patients. One of the principal challenges in the field of gene therapy is delivery of nucleic acid to target cells in a subject. Viral vectors have been extensively explored as delivery vehicles and have proven to be effective in certain circumstances, but viral vectors have been associated with several drawbacks, including immunogenicity. Consequently, non-viral approaches to gene therapy delivery are emerging as a promising alternative. Thus, a need exists for improved polypeptides capable of delivering nucleic acids to target cells.

### SUMMARY OF THE INVENTION

Provided herein are polypeptides and compositions containing the same that are useful in the delivery of therapeutic agents (e.g., nucleic acid vectors) to target cells. The polypeptides described herein can associate with nucleic acids as a pharmaceutical composition and disassociate from them after entry into a target cell (e.g., after entering the cell and/or nucleus) to facilitate expression of the nucleic acid by the target cell.

In one aspect, the invention features a polypeptide that includes [A]-[B], wherein [A] is a DNA condensing polypeptide; and [B] is a nuclear localization sequence (NLS).

In another aspect, the invention features a composition that includes a polypeptide that includes [A]-[B], wherein [A] is a DNA condensing polypeptide; and [B] is a nuclear localization sequence (NLS). The composition further includes a polynucleotide.

In another aspect, the invention features a composition that includes a polypeptide that includes [A]-[B], wherein [A] is a DNA condensing polypeptide; and [B] is a nuclear localization sequence (NLS).

The composition further includes a lipid.

In another aspect, the invention features a composition that includes a polypeptide that includes [A]-[B], wherein [A] is a DNA condensing polypeptide; and [B] is a nuclear localization sequence (NLS). The composition further includes a polynucleotide and a lipid. In some embodiments, the polypeptide is from 2 kDa to 5 kDa (e.g., 2 kDa, 2.5 kDa, 3 kDa, 3.5 kDa, 4 kDa, 4.5 kDa, or 5 kDa).

In some embodiments, the polypeptide is from 20 to 50 (e.g., 20 to 45, 20 to 40, 25 to 50, 25 to 40, 30 to 50, 30 to 45, or 35 to 50, e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acid residues in length.

In some embodiments, 30-50% (e.g., 30%-35%, 30%-40%, 40-45%, or 45-50%, e.g., 30%, 31%, 32%, 33%, 34%, 35%, 36%, 37%, 38%, 39%, 40%, 41%, 42%, 43%, 44%, 45%, 46%, 47%, 48%, 49%, or 50%) of the residues of the polypeptide are cationic.

In some embodiments, the NLS is seven, eight, or nine amino acid residues in length. The NLS may include, for example, a simian virus 40 (SV40) NLS or a c-MYC NLS. The NLS may include the amino acid sequence PKKKRKV (SEQ ID NO: 1). The NLS may include the amino acid sequence PAAKRVKL (SEQ ID NO: 2). The NLS may include the amino acid sequence of PAAKRVKLD (SEQ ID NO: 3) or VKRKKKP (SEQ ID NO: 4).

In some embodiments, the polypeptide includes a linker between [A] and [B]. The linker may be, for example, from two to 20 (e.g., 2 to 8, e.g., 2, 3, 4, 5, 6, 7, 8, 9, 10, 11, 12, 13, 14, 15, 16, 17, 18, 19, or 20, e.g., 4 or 5) amino acid residues in length. The linker may be a flexible linker. The linker may contain one or more glycines and/or serines. For example, the linker may have or include the amino acid sequence of GGGS (SEQ ID NO: 5), KSGG (SEQ ID NO: 6), CGGGS (SEQ ID NO: 7), or CGGS, (SEQ ID NO: 8). In some embodiments, the linker is a rigid linker, e.g., that substantially constrains the DNA condensing polypeptide (e.g., an alpha helix contained therein) and the NLS relative to each other.

In some embodiments, the DNA condensing polypeptide includes an amphipathic alpha helix. The amphipathic alpha helix may be, e.g., from 12 to 42 (e.g., 12 to 40, 15 to 40, 15 to 35, 20 to 40, 20 to 30, 25 to 40, or 30 to 40, e.g., 20-30, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42) amino acid residues in length. In some embodiments, the amphipathic alpha helix is from 20 to 30 amino acid residues in length.

In some embodiments, the amphipathic alpha helix includes a RALA motif. In some embodiments, the amphipathic alpha helix includes a plurality of RALA motifs. For example, the amphipathic alpha helix may include two, three, four, five, or more RALA motifs. In some embodiments, the amphipathic alpha helix includes three RALA motifs.

In some embodiments, the polypeptide includes an amino acid sequence having at least 85%, 90%, 95%, 97%, or 99% sequence identity to WEARLARALARALARHLARALARALRACEA (SEQ ID NO: 9). For example, the polypeptide may include the amino acid sequence of WEARLARALARALARHLARALARALRACEA (SEQ ID NO: 9). In some embodiments, the polypeptide includes an amino acid sequence having at least 85%, 90%, 95%, 97%, or 99% sequence identity to WEARLARALARALARHLARALARALRACEAPKKKRKV (SEQ ID NO: 10). For example, the polypeptide may include the amino acid sequence of WEARLARALARALARHLARALARALRACEAPKKKRKV (SEQ ID NO: 10).

In some embodiments, the DNA condensing polypeptide includes a DNA binding domain. The DNA binding domain may include an alpha helix. In some embodiments, the alpha helix is from 12 to 42 (e.g., 12 to 40, 15 to 40, 15 to 35, 20 to 40, 20 to 30, 25 to 40, or 30 to 40, e.g., 20-30, e.g., 12, 13, 14, 15,16,17,18,19, 20, 21, 22, 23, 24, 25, 26, 27, 28, 29, 30, 31, 32, 33, 34, 35, 36, 37, 38, 39, 40, 41, or 42) residues. In some embodiments, the alpha helix is from 20 to 30 amino acid residues in length.

In some embodiments, the alpha helix is non-amphipathic. The non-amphipathic alpha helix may have a hydrophobic moment (pH) of less than 1.0.

In some embodiments, the polypeptide includes an amino acid sequence of KARKKKLNKKGRKMAGRKRGR (SEQ ID NO: 11) or a variant thereof including an amino acid sequence that differs from KARKKKLNKKGRKMAGRKRGR (SEQ ID NO: 11) by no more than 6 amino acids (e.g., no more than 5, no more than 4, no more than 3, no more than 2, or no more than 1 amino acid). For example, the polypeptide may include the amino acid sequence of KARKX₁KLX₂X₃KGRX₄MAGRKRGR (SEQ ID NO: 12), wherein X₁, X₂, X₃, and X₄ are each, independently, any amino acid. In some embodiments, X₁, X₂, X₃, and X₄ are each, independently, selected from lysine, alanine, asparagine, arginine, and leucine. In some embodiments, the polypeptide includes the amino acid sequence of KARKAKLRLKGRLMAGRKRGR (SEQ ID NO: 13).

In some embodiments, the polypeptide includes an amino acid sequence having at least 85%, 90%, 95%, 97%, or 99% sequence identity to KARKKKLNKKGRKMAGRKRGR (SEQ ID NO: 11), KARKKKLNKKGRKMAGRKRGRPK (SEQ ID NO: 14), KARKAKLRLKGRLMAGRKRGRPK (SEQ ID NO: 15), KARKAKLRLKGRLMAGRKRGRP (SEQ ID NO: 16), KARKKKLNKKGRKMAGRKRGRP (SEQ ID NO: 17), KARKAKLRLKARLWARHRARACEA (SEQ ID NO: 18), or KARKAKLRLKGRLWARHRACEA (SEQ ID NO: 19).

In some embodiments, the polypeptide includes the amino acid sequence of

(SEQ ID NO: 11)
KARKKKLNKKGRKMAGRKRGR,
(SEQ ID NO: 14)
KARKKKLNKKGRKMAGRKRGRPK,
(SEQ ID NO: 15)
KARKAKLRLKGRLMAGRKRGRPK,
(SEQ ID NO: 16)
KARKAKLRLKGRLMAGRKRGRP,
(SEQ ID NO: 17)
KARKKKLNKKGRKMAGRKRGRP,
(SEQ ID NO: 18)
KARKAKLRLKARLWARHRARACEA,
or
(SEQ ID NO: 19)
KARKAKLRLKGRLWARHRACEA.

In some embodiments, the polypeptide includes an amino acid sequence having at least 85%, 90%, 95%, 97%, or 99% sequence identity to KARKKKLNKKGRKMAGRKRGRPKKKRKV (SEQ ID NO: 20), KARKKKLNKKGRKMAGRKRGRPKGGGSPAAKRVKLD (SEQ ID NO: 21), KARKAKLRLKGRLMAGRKRGRPKKSGGVKRKKKP (SEQ ID NO: 22), KARKAKLRLKGRLMAGRKRGRPKGGGSPAAKRVKLD (SEQ ID NO: 23), KARKAKLRLKGRLMAGRKRGRPCGGGSPAAKRVKL (SEQ ID NO: 24), KARKAKLRLKGRLMAGRKRGRPCGGSPAAKRVKL (SEQ ID NO: 25), KARKKKLNKKGRKMAGRKRGRPCGGSPKKKRKV (SEQ ID NO: 26), KARKAKLRLKGRLMAGRKRGRPCGGSPKKKRKV (SEQ ID NO: 27), KARKAKLRLKARLWARHRARACEAPAAKRVKL (SEQ ID NO: 28), KARKAKLRLKGRLWARHRACEAPAAKRVKL (SEQ ID NO: 29), or KARKAKLRLKGRLWARHRACEAPKKKRKV (SEQ ID NO: 30).

In some embodiments, the polypeptide includes the amino acid sequence of

(SEQ ID NO: 20)
KARKKKLNKKGRKMAGRKRGRPKKKRKV,
(SEQ ID NO: 21)
KARKKKLNKKGRKMAGRKRGRPKGGGSPAAKRVKLD,
(SEQ ID NO: 22)
KARKAKLRLKGRLMAGRKRGRPKKSGGVKRKKKP,
(SEQ ID NO: 23)
KARKAKLRLKGRLMAGRKRGRPKGGGSPAAKRVKLD,
(SEQ ID NO: 24)
KARKAKLRLKGRLMAGRKRGRPCGGGSPAAKRVKL,
(SEQ ID NO: 25)
KARKAKLRLKGRLMAGRKRGRPCGGSPAAKRVKL,
(SEQ ID NO: 26)
KARKKKLNKKGRKMAGRKRGRPCGGSPKKKRKV,
(SEQ ID NO: 27)
KARKAKLRLKGRLMAGRKRGRPCGGSPKKKRKV,
(SEQ ID NO: 28)
KARKAKLRLKARLWARHRARACEAPAAKRVKL,
(SEQ ID NO: 29)
KARKAKLRLKGRLWARHRACEAPAAKRVKL,
or
(SEQ ID NO: 30)
KARKAKLRLKGRLWARHRACEAPKKKRKV.

In some embodiments, the polynucleotide is from about 500 nucleotides to about 20,000 nucleotides (e.g., from about 500 to about 1,000, e.g., about 500, 600, 700, 800, 900, or 1,000, e.g., from about 1,000 to about 2,000, e.g., about 1,100, 1,200, 1,300, 1,400, 1,500, 1,600, 1,700, 1,800, 1,900, or 2,000, e.g., from about 2,000 to about 20,000, e.g., about 3,000, 4,000, 5,000, 6,000, 7,000, 8,000, 9,000, 10,000, 11,000, 12,000, 13,000, 14,000, 15,000, 16,000, 17,000, 18,000, 19,000, or 20,000) nucleotides in length.

In some embodiments, the polynucleotide encodes a protein.

In some embodiments, the polynucleotide is a non-viral polynucleotide.

In some embodiments, the polynucleotide is a closed circular polynucleotide.

In some embodiments, the polynucleotide is DNA. For example, the polynucleotide may be a closed circular supercoiled DNA.

In some embodiments, the polypeptide and the polynucleotide are present at a molar ratio of from about 100:1 to about 10,000,000:1, e.g., a molar ratio of from about 1,000:1 to about 10,000:1 (e.g., about 1,000:1, 2,000:1, 3,000:1, 4,000:1, 5,000:1, 6,000:1, 7,000:1, 8,000:1, 9,000:1, or 10,000:1.

In some embodiments, the lipid is a phospholipid.

In some embodiments, the lipid is a cationic lipid. In some embodiments, the cationic lipid is 4-(dimethylamino)-butanoic acid, (10Z,13Z)-1-(9Z,12Z)-9,12-octadecadien-1-yl-10,13-nonadecadien-1-yl ester (DLin-MC3-DMA), 1,2-dioleoyl-3-dimethylammonium-propane (DODAP), 1,2-dioleyloxy-3-dimethylaminopropane (DODMA), N,N-dimethyl-2,2-di-(9Z,12Z)-9,12-octadecadien-1-yl-1,3-dioxolane-4-ethanamine (DLin-KC2-DMA), 4-(dimethylamino)-butanoic acid, (10Z,13Z)-1-(9Z,12Z)-9,12-octadecadien-1-yl-10,13-nonadecadien-1-yl ester (DLin-MC3-DMA), OF-02, 3,6-bis({4-[bis(2-hydroxydodecyl)amino]butyl})piperazine-2,5-dione (CKK-E12), 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), or 2-(dioctylamino)ethyl nonyl hydrogen phosphate (9A1P9).

In some embodiments, the lipid is a PEGylated lipid. In some embodiments, the PEGylated lipid is 1,2-Dimyristoyl-sn-glycero-3-methoxypolyethylene glycol (DMG-PEG). For example, the DMP-PEG may be DMG-PEG 2000.

In some embodiments, the lipid is an anionic or neutral lipid. In some embodiments, the lipid is an anionic lipid. In some embodiments, the lipid is a neutral lipid. In some embodiments, the neutral lipid is 1,2-Dioleoyl-sn-glycero-3-phosphoethanolamine (DOPE).

In some embodiments, the lipid is a sterol. The sterol may be, for example, cholesterol or a derivative thereof.

In some embodiments, the composition includes a mixture of lipids. For example, in some embodiments, the composition includes a cationic lipid, a neutral lipid, an anionic lipid, a PEGylated lipid, a sterol, or any combination or variations thereof. In some embodiments, the composition includes a cationic lipid, a neutral lipid, a PEGylated lipid, and a sterol. In some embodiments, the composition includes DLin-MC3-DMA, DOPE, cholesterol, DMG-PEG 2000, or a combination thereof. In some embodiments, the composition includes a mixture of DLin-MC3-DMA, DOPE, cholesterol, and DMG-PEG 2000. The mixture may include DLin-MC3-DMA, DOPE, cholesterol, and DMG-PEG 2000 at a molar ratio of about 40-60:15-20:25-35:1-2. For example, the mixture may include DLin-MC3-DMA, DOPE, cholesterol, and DMG-PEG 2000 at a molar ratio of about 50:18.5:30:1.5.

In some embodiments, the composition includes the polypeptide and the polynucleotide at a molar ratio of from about 1:1 to about 10,000,000:1 (e.g., from about 1,000:1 to about 10,000:1, e.g., from about 1,000:1 to about 7,000:1, e.g., about 1,500:1, 2,000:1, 2,500:1, 3,000:1, 3,500:1, 4,000:1, 4,500:1, 5,000:1, 5,500:1, 6,000:1, 6,500:1, 7,000:1, 7,500:1, 8,000:1, 8,500:1, 9,000:1, 9,500:1, or 10,000:1, e.g., about 1,500:1, about 3,000:1, or about 6,000:1.

In some embodiments, the composition includes a nanoparticle including the polypeptide, the polynucleotide, and the lipid.

In some embodiments, the composition includes a plurality of the polypeptides, a plurality of the polynucleotides, and a plurality of the lipids. The composition may include a plurality of nanoparticles. In some embodiments, at least 90% of the nanoparticles in the composition have a diameter from about 10 nm to about 500 nm (e.g., from about 10 nm to about 250 nm, e.g., about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, or 500 nm) as measured by dynamic light scattering (DLS).

In another aspect, the invention features a method of introducing a polynucleotide into a target cell by contacting the target cell with a composition as described herein. The target cell may be, for example, a mammalian cell (e.g., a human cell). The contacting may be performed by in vivo administration of the composition to a subject that has the target cell. Alternatively, the contacting may be performed in vitro. Following the in vitro contacting, the target cell may be introduced into the subject.

In some embodiments, the polynucleotide is expressed by the target cell. The expression may be measured by detecting a protein encoded by the polynucleotide. In some embodiments, the composition is less immunogenic than a reference composition without the polypeptide (e.g., as measured by cGAS or STING signaling). In some embodiments, cGAS and/or STING signaling is absent or undetectable in response to the composition contacting the target cell.

In another aspect, the invention features a polypeptide that includes [C]-[L]-[D], wherein [C] is a non-amphipathic alpha helix from 12 to 42 amino acid residues in length; [L] is a linker from one to 20 amino acid residues in length or is absent; and [D] is a nuclear localization sequence (NLS) from five to 12 amino acid residues in length.

In another aspect, the invention features a polypeptide that includes [C]-[L]-[D], wherein [C] is a non-amphipathic alpha helix from 12 to 42 amino acid residues in length; [L] is a linker from two to 20 amino acid residues in length; and [D] is a nuclear localization sequence (NLS) from five to 12 amino acid residues in length.

In another aspect, the invention features a polypeptide that includes [C]-[D], wherein [C] is a non-amphipathic alpha helix from 12 to 42 amino acid residues in length; and [D] is a nuclear localization sequence (NLS) from five to 12 amino acid residues in length.

In another aspect, the invention features a polypeptide that includes [C]-[L]-[D], wherein [C] is a non-amphipathic alpha helix from 12 to 42 amino acid residues in length; [L] is a linker of 1 or 2 amino acid residues in length; and [D] is a nuclear localization sequence (NLS) from five to 12 amino acid residues in length.

The linker may be, for example, from two to 20 (e.g., 2 to 8, e.g., 2, 3, 4, 5, 6, 7, 8, 9, 10, 11, 12, 13, 14, 15, 16, 17, 18, 19, or 20, e.g., 4 or 5) amino acid residues in length. The linker may be one amino acid residue. The linker may be a flexible linker. The linker may contain one or more glycines and/or serines. For example, the linker may have or include the amino acid sequence of GGGS (SEQ ID NO: 5), KSGG (SEQ ID NO: 6), CGGGS (SEQ ID NO: 7), or CGGS, (SEQ ID NO: 8). In some embodiments, the linker is a rigid linker, e.g., that substantially constrains the alpha helix and the NLS relative to each other.

In some embodiments, the NLS is seven, eight, or nine amino acid residues in length. The NLS may include, for example, a simian virus 40 (SV40) NLS or a c-MYC NLS. The NLS may include the amino acid sequence PKKKRKV (SEQ ID NO: 1). The NLS may include the amino acid sequence PAAKRVKL (SEQ ID NO: 2). The NLS may include the amino acid sequence of PAAKRVKLD (SEQ ID NO: 3) or VKRKKKP (SEQ ID NO: 4).

In some embodiments, the non-amphipathic alpha helix is from 12 to 42 (e.g., 12 to 40, 15 to 40, 15 to 35, 20 to 40, 20 to 30, 25 to 40, or 30 to 40, e.g., 20-30, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42) residues. In some embodiments, the non-amphipathic alpha helix is from 20 to 30 amino acid residues in length. The non-amphipathic alpha helix may have a hydrophobic moment (pH) of less than 1.0.

In some embodiments, the non-amphipathic alpha helix includes an amino acid sequence of KARKKKLNKKGRKMAGRKRGR (SEQ ID NO: 11) or a variant thereof including an amino acid sequence that differs from KARKKKLNKKGRKMAGRKRGR (SEQ ID NO: 11) by no more than 6 amino acids (e.g., no more than 5, no more than 4, no more than 3, no more than 2, or no more than 1 amino acid). In some embodiments, the non-amphipathic alpha helix includes the amino acid sequence of KARKX₁KLX₂X₃KGRX₄MAGRKRGR (SEQ ID NO: 12), wherein X₁, X₂, X₃, and X₄ are each, independently, any amino acid. In some embodiments, X₁, X₂, X₃, and X₄ are each, independently, selected from lysine, alanine, asparagine, arginine, and leucine. In some embodiments, the polypeptide includes the amino acid sequence of KARKAKLRLKGRLMAGRKRGR (SEQ ID NO: 13).

In some embodiments, the non-amphipathic alpha helix includes an amino acid sequence having at least 85%, 90%, 95%, 97%, or 99% sequence identity to KARKKKLNKKGRKMAGRKRGR (SEQ ID NO: 11), KARKKKLNKKGRKMAGRKRGRPK (SEQ ID NO: 14), KARKAKLRLKGRLMAGRKRGRPK (SEQ ID NO: 15), KARKAKLRLKGRLMAGRKRGRP (SEQ ID NO: 16), KARKKKLNKKGRKMAGRKRGRP (SEQ ID NO: 17), KARKAKLRLKARLWARHRARACEA (SEQ ID NO: 18), or KARKAKLRLKGRLWARHRACEA (SEQ ID NO: 19).

In some embodiments, the non-amphipathic alpha helix includes the amino acid sequence of

(SEQ ID NO: 11)
KARKKKLNKKGRKMAGRKRGR,
(SEQ ID NO: 14)
KARKKKLNKKGRKMAGRKRGRPK,
(SEQ ID NO: 15)
KARKAKLRLKGRLMAGRKRGRPK,
(SEQ ID NO: 16)
KARKAKLRLKGRLMAGRKRGRP,
(SEQ ID NO: 17)
KARKKKLNKKGRKMAGRKRGRP,
(SEQ ID NO: 18)
KARKAKLRLKARLWARHRARACEA,
or
(SEQ ID NO: 19)
KARKAKLRLKGRLWARHRACEA.

In some embodiments, the polypeptide includes an amino acid sequence having at least 85%, 90%, 95%, 97%, or 99% sequence identity to KARKKKLNKKGRKMAGRKRGRPKKKRKV (SEQ ID NO: 20), KARKKKLNKKGRKMAGRKRGRPKGGGSPAAKRVKLD (SEQ ID NO: 21), KARKAKLRLKGRLMAGRKRGRPKKSGGVKRKKKP (SEQ ID NO: 22), KARKAKLRLKGRLMAGRKRGRPKGGGSPAAKRVKLD (SEQ ID NO: 23), KARKAKLRLKGRLMAGRKRGRPCGGGSPAAKRVKL (SEQ ID NO: 24), KARKAKLRLKGRLMAGRKRGRPCGGSPAAKRVKL (SEQ ID NO: 25), KARKKKLNKKGRKMAGRKRGRPCGGSPKKKRKV (SEQ ID NO: 26), KARKAKLRLKGRLMAGRKRGRPCGGSPKKKRKV (SEQ ID NO: 27), KARKAKLRLKARLWARHRARACEAPAAKRVKL (SEQ ID NO: 28), KARKAKLRLKGRLWARHRACEAPAAKRVKL (SEQ ID NO: 29), or KARKAKLRLKGRLWARHRACEAPKKKRKV (SEQ ID NO: 30).

In some embodiments, the polypeptide includes the amino acid sequence of

(SEQ ID NO: 20)
KARKKKLNKKGRKMAGRKRGRPKKKRKV,
(SEQ ID NO: 21)
KARKKKLNKKGRKMAGRKRGRPKGGGSPAAKRVKLD,
(SEQ ID NO: 22)
KARKAKLRLKGRLMAGRKRGRPKKSGGVKRKKKP,
(SEQ ID NO: 23)
KARKAKLRLKGRLMAGRKRGRPKGGGSPAAKRVKLD,
(SEQ ID NO: 24)
KARKAKLRLKGRLMAGRKRGRPCGGGSPAAKRVKL,
(SEQ ID NO: 25)
KARKAKLRLKGRLMAGRKRGRPCGGSPAAKRVKL,
(SEQ ID NO: 26)
KARKKKLNKKGRKMAGRKRGRPCGGSPKKKRKV,
(SEQ ID NO: 27)
KARKAKLRLKGRLMAGRKRGRPCGGSPKKKRKV,
(SEQ ID NO: 28)
KARKAKLRLKARLWARHRARACEAPAAKRVKL,
(SEQ ID NO: 29)
KARKAKLRLKGRLWARHRACEAPAAKRVKL,
or
(SEQ ID NO: 30)
KARKAKLRLKGRLWARHRACEAPKKKRKV.

In some embodiments, the polypeptide is from 2 kDa to 5 kDa (e.g., 2 kDa, 2.5 kDa, 3 kDa, 3.5 kDa, 4 kDa, 4.5 kDa, or 5 kDa).

In some embodiments, the polypeptide is from 20 to 50 (e.g., 20 to 45, 20 to 40, 25 to 50, 25 to 40, 30 to 50, 30 to 45, or 35 to 50, e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acid residues in length.

In some embodiments, 30-50% (e.g., 30%-35%, 30%-40%, 40-45%, or 45-50%, e.g., 30%, 31%, 32%, 33%, 34%, 35%, 36%, 37%, 38%, 39%, 40%, 41%, 42%, 43%, 44%, 45%, 46%, 47%, 48%, 49%, or 50%) of the residues of the polypeptide are cationic.

In another aspect, the invention features a composition that includes a polypeptide as described herein and a polynucleotide.

In some embodiments, the polynucleotide is from about 500 nucleotides to about 20,000 nucleotides (e.g., from about 500 to about 1,000, e.g., about 500, 600, 700, 800, 900, or 1,000, e.g., from about 1,000 to about 2,000, e.g., about 1,100, 1,200, 1,300, 1,400, 1,500, 1,600, 1,700, 1,800, 1,900, or 2,000, e.g., from about 2,000 to about 20,000, e.g., about 3,000, 4,000, 5,000, 6,000, 7,000, 8,000, 9,000, 10,000, 11,000, 12,000, 13,000, 14,000, 15,000, 16,000, 17,000, 18,000, 19,000, or 20,000) nucleotides in length.

In some embodiments, the polynucleotide encodes a protein.

In some embodiments, the polynucleotide is a non-viral polynucleotide.

In some embodiments, the polynucleotide is a closed circular polynucleotide.

In some embodiments, the polynucleotide is DNA. For example, the polynucleotide may be a closed circular supercoiled DNA.

In some embodiments, the polypeptide and the polynucleotide are present at a molar ratio of from about 100:1 to about 10,000,000:1, e.g., a molar ratio of from about 1,000:1 to about 10,000:1 (e.g., about 1,000:1, 2,000:1, 3,000:1, 4,000:1, 5,000:1, 6,000:1, 7,000:1, 8,000:1, 9,000:1, or 10,000:1.

In some embodiments, the composition further includes a lipid. For example, the composition may include a mixture of lipids.

In some embodiments, the lipid is a phospholipid.

In some embodiments, the lipid is a cationic lipid. In some embodiments, the cationic lipid is 4-(dimethylamino)-butanoic acid, (10Z,13Z)-1-(9Z,12Z)-9,12-octadecadien-1-yl-10,13-nonadecadien-1-yl ester (DLin-MC3-DMA), 1,2-dioleoyl-3-dimethylammonium-propane (DODAP), 1,2-dioleyloxy-3-dimethylaminopropane (DODMA), N,N-dimethyl-2,2-di-(9Z,12Z)-9,12-octadecadien-1-yl-1,3-dioxolane-4-ethanamine (DLin-KC2-DMA), 4-(dimethylamino)-butanoic acid, (10Z,13Z)-1-(9Z,12Z)-9,12-octadecadien-1-yl-10,13-nonadecadien-1-yl ester (DLin-MC3-DMA), OF-02, 3,6-bis({4-[bis(2-hydroxydodecyl)amino]butyl})piperazine-2,5-dione (CKK-E12), 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), or 2-(dioctylamino)ethyl nonyl hydrogen phosphate (9A1P9).

In some embodiments, the lipid is a PEGylated lipid. In some embodiments, the PEGylated lipid is 1,2-Dimyristoyl-sn-glycero-3-methoxypolyethylene glycol (DMG-PEG). For example, the DMP-PEG may be DMG-PEG 2000.

In some embodiments, the lipid is an anionic or neutral lipid. In some embodiments, the lipid is an anionic lipid. In some embodiments, the lipid is a neutral lipid. In some embodiments, the neutral lipid is 1,2-Dioleoyl-sn-glycero-3-phosphoethanolamine (DOPE).

In some embodiments, the lipid is a sterol. The sterol may be, for example, cholesterol or a derivative thereof.

In some embodiments, the composition includes a mixture of lipids. For example, in some embodiments, the composition includes a cationic lipid, a neutral lipid, an anionic lipid, a PEGylated lipid, a sterol, or any combination or variations thereof. In some embodiments, the composition includes a cationic lipid, a neutral lipid, a PEGylated lipid, and a sterol. In some embodiments, the composition includes DLin-MC3-DMA, DOPE, cholesterol, DMG-PEG 2000, or a combination thereof. In some embodiments, the composition includes a mixture of DLin-MC3-DMA, DOPE, cholesterol, and DMG-PEG 2000. The mixture may include DLin-MC3-DMA, DOPE, cholesterol, and DMG-PEG 2000 at a molar ratio of about 40-60:15-20:25-35:1-2. For example, the mixture may include DLin-MC3-DMA, DOPE, cholesterol, and DMG-PEG 2000 at a molar ratio of about 50:18.5:30:1.5.

In some embodiments, the composition includes a cationic lipid, a neutral lipid, a sterol, and a PEGylated lipid at a molar ratio of about 40-60:15-20:25-35:1-2. For example, the mixture may include a cationic lipid, a neutral lipid, a sterol, and a PEGylated lipid at a molar ratio of about 50:18.5:30:1.5.

In some embodiments, the composition includes a cationic lipid (e.g., DLin-MC3-DMA) and a neutral lipid (e.g., DOPE) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4,1:5, 1:6, 1:7,1:8, 1:9, 1:10, 1:15, 1:20, 1:25, 1:30, 1:35, 1:40, 1:45, 1:50: 1:60, 1:70, 1:80, 1:90, or 1:100).

In some embodiments, the composition includes a cationic lipid (e.g., DLin-MC3-DMA) and a sterol (e.g., cholesterol) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the composition includes a cationic lipid (e.g., DLin-MC3-DMA) and a PEGylated lipid (e.g., DMG-PEG) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the composition includes a neutral lipid (e.g., DOPE) and a sterol (e.g., cholesterol) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the composition includes a neutral lipid (e.g., DOPE) and a PEGylated lipid (e.g., DMG-PEG) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the composition includes a sterol (e.g., cholesterol and a PEGylated lipid (e.g., DMG-PEG) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the composition includes a nanoparticle that includes the polypeptide, the polynucleotide, and the lipid. In some embodiments, the composition includes a plurality of the polypeptides, a plurality of the polynucleotides, and a plurality of the lipids. In some embodiments, the composition includes a plurality of the polypeptides, a plurality of the polynucleotides, and a plurality of the lipids. The composition may include a plurality of nanoparticles. In some embodiments, at least 90% of the nanoparticles in the composition have a diameter from about 10 nm to about 500 nm (e.g., from about 10 nm to about 250 nm, e.g., about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, or 500 nm) as measured by dynamic light scattering (DLS).

In another aspect, the invention features a method of introducing a polynucleotide into a target cell by contacting the target cell with a composition as described herein. The target cell may be, for example, a mammalian cell (e.g., a human cell). The contacting may be performed by in vivo administration of the composition to a subject that has the target cell. Alternatively, the contacting may be performed in vitro. Following the in vitro contacting, the target cell may be introduced into the subject.

In some embodiments, the polynucleotide is expressed by the target cell. The expression may be measured by detecting a protein encoded by the polynucleotide. In some embodiments, the composition is less immunogenic than a reference composition without the polypeptide (e.g., as measured by cGAS or STING signaling). In some embodiments, cGAS and/or STING signaling is absent or undetectable in response to the composition contacting the target cell.

### Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, the term “about” refers to a value within ±10% variability from the reference value, unless otherwise specified.

As used herein, the term “alpha-helix” refers to a right-handed coiled or spiral conformation having an angle of rotation between consecutive amino acids of about 100 degrees and/or 3.6 residues per turn. Methods of predicting the presence of alpha-helices in proteins and polypeptides are well-known in the art, for example, based on 3D modeling using PEP-FOLD (bioserv.rpbs.univ-paris-diderot.ff/services/PEP-FOLD/).

As used herein, the term “non-amphipathic alpha-helix” refers to an alpha-helix having a hydrophobic moment (pH) of less than 3.5, calculated as described in Eisenberg et al., Nature 299: 371-374, 1982.

As used herein, amino acid residues containing a “hydrophobic side chains” include alanine (A), isoleucine (I), leucine (L), methionine (M), phenylalanine (F), tryptophan (W), tyrosine (Y), and valine (V).

As used herein, amino acid residues containing a “positively charged side chain” refer to amino acid residues having a positively charged side chain at physiological conditions, which include arginine (R), histidine (H), and lysine (K).

As used herein, “polypeptide,” “peptide,” or “protein” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or a multi-molecular complex such as a dimer, trimer, or tetramer. They can also include single chain or multichain polypeptides can be associated or linked. The term polypeptide can also apply to amino acid polymers in which one or more (e.g., two or more) amino acid residues may be an artificial chemical analogue of a corresponding naturally occurring amino acid.

The term “DNA condensing polypeptide” refers to a polypeptide or domain thereof that forms particles in solution upon mixing with DNA, e.g., as measured by dynamic light scattering (DLS).

The term “polynucleotide,” as used herein, means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

The term “non-viral polynucleotide” means a polynucleotide that is devoid of components inherent to viral vectors (e.g., viral proteins).

The term “closed circular” or “covalently closed circular” means polynucleotide in a circular form that lacks free 5′ and 3′ free ends.

As used herein, the term “sequence identity” is determined by alignment of two peptide or two nucleotide sequences using a global or local alignment algorithm. Sequences are referred to as “substantially identical” or “essentially similar” when they share at least a certain minimal percentage of sequence identity when optimally aligned (e.g., when aligned by programs such as GAP or BESTFIT using default parameters). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna, and for proteins the default scoring matrix is Blosum62 (Henikoff & Henikoff, 1992, PNAS 89, 915-919). Sequence alignments and scores for percentage sequence identity (e.g., 85%, 90%, 95%, 97%, 99%, or 100%) are determined, e.g., using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively, or additionally, percent identity is determined by searching against databases, e.g., using algorithms such as FASTA, BLAST, etc. Sequence identity refers to the sequence identity over the entire length of the sequence.

As used herein, “administering” is meant a method of giving a dosage of a composition (e.g., a polypeptide-polynucleotide composition, e.g., a particulate composition containing a polypeptide and polynucleotide) of the invention to an individual. The compositions utilized in the methods described herein can be administered intravenously, subcutaneously, intradermally, percutaneously, intramuscularly, intraarterially, intraperitoneally, intralesionally, intracranially, intraarticularly, intraprostatically, intrapleurally, intratracheally, intrathecally, intranasally, intravaginally, intrarectally, topically, peritoneally, subconjunctivally, intravesicularly, mucosally, intrapericardially, intraumbilically, orally, intraocularly (e.g., suprachoroidally, intravitreally, periocularly, or subretinally), topically, transdermally, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. The compositions utilized in the methods described herein can be administered systemically. The method of administration can vary depending on various factors (e.g., the compound or composition being administered and the severity of the condition, disease, or disorder being treated).

As used herein, “delivering,” “to deliver,” and grammatical variations thereof, is meant causing an agent (e.g., a polynucleotide) to access a target cell. The agent can be delivered by administration an individual having the target cell (e.g., systemically or locally administering the agent) such that the agent gains access to the organ or tissue in which the target cell resides. Additionally, or alternatively, the agent can be delivered by applying a stimulus to a tissue or organ harboring the agent, wherein the stimulus causes the agent to enter the target cell. Thus, in some instances, an agent is delivered to a target cell by transmitting an electric field into a tissue harboring the agent at conditions suitable for electrotransfer of the agent into a target cell within the tissue.

As used herein, “electrotransfer” refers to movement of a molecule (e.g., a polynucleotide) across a membrane of a target cell (e.g., from outside to inside the target cell, that is caused by transmission of an electric field (e.g., a pulsed electric field) to the microenvironment in which the cell resides. Electrotransfer may occur as a result of electrophoresis, i.e., movement of a molecule (e.g., a polynucleotide) along an electric field (e.g., in the direction of current), based on a charge of the molecule. Electrophoresis can induce electrotransfer, for example, by moving a molecule (e.g., a polynucleotide) into proximity of a cell membrane to allow a biotransport process (e.g., endocytosis including pinocytosis or phagocytosis) or passive transport (e.g., diffusion or lipid partitioning) to carry the molecule into the cell. Additionally, or alternatively, electrotransfer may occur as a result of electroporation, i.e., generation of pores in the target cell caused by transmission of an electric field (e.g., a pulsed electric field), wherein the size, shape, and duration of the pores are suitable to accommodate movement of a molecule (e.g., a nucleic acid, e.g., a naked nucleic acid) from outside the target cell to inside the target cell. Thus, in some instances, electrotransfer occurs as a result of a combination of electrophoresis and electroporation.

By “reduce or inhibit” is meant the ability to cause an overall decrease preferably of 20% or greater, more preferably of 50% or greater, and most preferably of 75%, 85%, 90%, 95%, or greater.

The terms “level of expression” or “expression level” are used interchangeably and generally refer to the amount of a polynucleotide or an amino acid product or protein in a biological sample (e.g., retina). “Expression” generally refers to the process by which gene-encoded information is converted into the structures present and operating in the cell. Therefore, according to the invention, “expression” of a gene may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, an “effective amount” or “effective dose” of a therapeutic agent (e.g., a polynucleotide) or composition thereof refers to an amount sufficient to achieve a desired biological and/or pharmacological effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat a disease may slow or stop disease progression or increase partial or complete response relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis.

In some embodiments, polynucleotides, and compositions thereof, of the invention are used to delay development of a disease or to slow the progression of a disease.

### BRIEF DESCRIPTION OF THE DRAWINGS

FIG. 1 shows an exemplary preparation of a DNA/polypeptide lipid nanoparticle (LNP). The composition includes a closed circular DNA vector, a cationic lipid, a PEGylated lipid, cholesterol, and a polypeptide with a nuclear localization sequence. The components are comixed in a microfluidic device, which allows lipid nanoparticles to form.

FIGS. 2A and 2B show agarose gel electrophoresis assays with various amounts of C3-fLuc1157 DNA encapsulated in LNPs. FIG. 2A shows various amounts of C3-fLuc1157 DNA encapsulated in LNPs; Group 2: LNP containing fLuc1157 mRNA; Group 3: LNP containing C3-fLuc1157; Group 4: LNP containing C3-fLuc1157 and RALA-NLS at 1150× molar ratio to C3 DNA. FIG. 2B shows an assessment for nucleic acid integrity and concentration as confirmed by payload release with SDS.

FIG. 3 is a graph showing normalized luciferase activity in HepG2 cells 48 hours after in vitro transfection. Lipid nanoparticles containing a polynucleotide encoding luciferase (fLuc1157) in the presence or absence of RALA-NLS polypeptides at various concentrations were used to transfect HepG2 cells in vitro. Group 1: PBS; Group 2: LNP containing mRNA-fLuc1157; Group 3: LNP containing covalently closed circular (C³) DNA encoding fLuc1157; Group 4: LNP containing C3-fLuc1157 and RALA-NLS at 1150× molar ratio to C3 DNA; Group 5: LNP containing C3-fLuc1157 and RALA-NLS at 3000× molar ratio to C3 DNA; Group 6: LNP containing C3-fLuc1157 and RALA-NLS at 6000× molar ratio to C3 DNA.

FIGS. 4A and 4B are graphs showing normalized luciferase activity in HepG2 cells 48 hours after in vitro transfection. Lipid nanoparticles were formed with RALA-NLS polypeptides and 250 ng or 500 ng C3-fLuc1157 DNA at a molar ratio of 700, 1400, 2100, or 2800. FIG. 4A shows LNPs containing DOPE. FIG. 4B shows LNPs containing 20% DOTAP; Lipo=Lipofectamine 3K positive control.

FIGS. 5A and 5B are graphs showing normalized luciferase activity in HepG2 cells 48 hours after in vitro transfection. Lipid nanoparticles were formed with RALA-NLS (RN) polypeptides (WEARLARALARALARHLARALARALRACEAPKKKRKV; SEQ ID NO: 10), DBD17-NLS (KARKAKLRLKARLWARHRARACEAPAAKRVKL; SEQ ID NO: 28), DBD18-NLS (KARKAKLRLKGRLWARHRACEAPAAKRVKL; SEQ ID NO: 29), or DBD19-NLS (KARKAKLRLKGRLWARHRACEAPKKKRKV; SEQ ID NO: 30). FIG. 5A shows LNPs. FIG. 5B shows polypeptide controls without LNP formulation; Lipo=Lipofectamine 3K positive control.

FIG. 6 is a graph showing normalized liver luciferase activity 48 hours and 96 hours after intravenous injection in mouse tail veins. Lipid nanoparticles containing a polynucleotide encoding luciferase (fLuc1157) in the presence or absence of RALA-NLS polypeptides (RALA-NLS) at various concentrations were used to transfect HepG2 cells in vivo. Group 1: PBS; Group 2: LNP containing mRNA-fLuc1157; Group 3: LNP containing C3-fLuc1157; Group 4: LNP containing C3-fLuc1157 and RALA-NLS at 1150× molar ratio to C3 DNA; Group 5: LNP containing C3-fLuc1157 and RALA-NLS at 3000× molar ratio to C3 DNA; Group 6: LNP containing C3-fLuc1157 and RALA-NLS at 6000× molar ratio to C3 DNA.

### DETAILED DESCRIPTION

Provided herein are polypeptides and compositions containing the same that are useful in the delivery of therapeutic agents (e.g., nucleic acid vectors) to target cells. In some instances, polypeptides described herein can associate with nucleic acids as a pharmaceutical composition and disassociate from them upon or after entry into a target cell (e.g., entry into the cell or entry into the nucleus of the cell) to facilitate expression of the nucleic acid by the target cell. Association of the polypeptides described herein with polynucleotides can reduce adverse effects (e.g., immunogenicity) of certain DNA vector compositions, e.g., by shielding the DNA vector from cytosolic signaling pathways, such as cyclic guanosine monophosphate-adenosine monophosphate synthase (cGAS) and/or stimulation of IFN genes (STING). In general, the invention features polypeptides containing a DNA condensing polypeptide and a nuclear localization sequence (NLS). The invention also features compositions containing a DNA condensing polypeptide, a polynucleotide, e.g., that associates with the polypeptide, and a lipid. The compositions can associate to form a lipid nanoparticle, which can be used for delivery of the polynucleotide to a target cell.

### Polypeptides

Provided herein are polypeptides that include [A]-[B], where [A] is a DNA condensing polypeptide, and [B] is a nuclear localization sequence (NLS). The DNA condensing polypeptide may include an alpha helix. The alpha helix may be, e.g., an amphipathic alpha helix or a non-amphipathic alpha helix. The DNA condensing polypeptide may contain a DNA binding domain. The DNA binding domain may contain an alpha helix (e.g., a non-amphipathic alpha helix).

Also provided herein are polypeptides that includes [C]-[L]-[D], where [C] is a non-amphipathic alpha helix from 12 to 42 amino acid residues in length; [L] is a linker from one to 20 amino acid residues in length or is absent; and [D] is a nuclear localization sequence (NLS) from five to 12 amino acid residues in length.

Also provided herein are polypeptides that includes [C]-[L]-[D], where [C] is a non-amphipathic alpha helix from 12 to 42 amino acid residues in length; [L] is a linker from two to 20 amino acid residues in length; and [D] is a nuclear localization sequence (NLS) from five to 12 amino acid residues in length.

In some embodiments, the polypeptide is from 2 kDa to 5 kDa (e.g., 2 kDa, 2.5 kDa, 3 kDa, 3.5 kDa, 4 kDa, 4.5 kDa, or 5 kDa).

In some embodiments, the polypeptide is from 20 to 50 (e.g., 20 to 45, 20 to 40, 25 to 50, 25 to 40, 30 to 50, 30 to 45, or 35 to 50, e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acid residues in length.

In some embodiments, 30-50% (e.g., 30%-35%, 30%-40%, 40-45%, or 45-50%, e.g., 30%, 31%, 32%, 33%, 34%, 35%, 36%, 37%, 38%, 39%, 40%, 41%, 42%, 43%, 44%, 45%, 46%, 47%, 48%, 49%, or 50%) of the residues of the polypeptide are cationic.

In some embodiments, the alpha helix (e.g., non-amphipathic alpha-helix) has a net positive charge capable of promoting non-covalent binding with a polynucleotide, such as DNA or RNA, e.g., under physiological conditions. In some instances, the alpha helix has a net charge from +4 to +16 (e.g., from +6 to +15, from +8 to +14, from +10 to +13; e.g., from +8 to +10, from +10 to +12, from +12 to +14, or from +14 to +16; e.g., +4, +5, +6, +7, +8, +9, +10, +11, +12, +13, +14, +15, or +16), e.g., under physiological conditions. In some instances, the alpha helix has a net charge from +8 to +16 (e.g., from +9 to +15, from +10 to +14, from +11 to +13; e.g., from +8 to +10, from +10 to +12, from +12 to +14, or from +14 to +16; e.g., +8, +9, +10, +11, +12, +13, +14, +15, or +16), e.g., under physiological conditions.

In some embodiments, the alpha-helix includesl2 to 42 (e.g., 12 to 40, 15 to 40, 15 to 35, 20 to 40, 20 to 30, 25 to 40, or 30 to 40, e.g., 20-30, e.g., 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42) residues. In some embodiments, the alpha-helix is 16 to 30 amino acid residues in length (e.g., from 17 to 28 amino acid residues in length, from 18 to 26 amino acid residues in length, from 19 to 24 amino acid residues in length, or from amino acid 20 to 22 residues in length (e.g., 21 amino acid residues in length)).

In some embodiments, at least 20% of the residues of the non-amphipathic alpha-helix have hydrophobic side chains (e.g., 20% to 40%, 20% to 35%, 20% to 30%, 25% to 40%, 25% to 35%, 25% to 30%, 30% to 40%, or 30% to 35% of the residues of the non-amphipathic alpha-helix have hydrophobic side chains). In some embodiments, at least five out of 21 consecutive residues of the non-amphipathic alpha-helix have hydrophobic side chains. In some embodiments, at least 20% of the residues of the non-amphipathic alpha-helix are selected from alanine, leucine, and methionine (e.g., 20% to 40%, 20% to 35%, 20% to 30%, 25% to 40%, 25% to 35%, 25% to 30%, 30% to 40%, or 30% to 35% of the residues of the non-amphipathic alpha helix are selected from alanine, leucine, and methionine). In some embodiments, at least five out of 21 consecutive residues of the non-amphipathic alpha-helix are selected from alanine, leucine, and methionine.

In some embodiments, 40% to 60% of the residues of the non-amphipathic alpha-helix are positively charged (e.g., 45% to 55%, e.g., about 50% of the residues of the non-amphipathic alpha-helix are positively charged) (e.g., selected from arginine, histidine, and lysine). In some embodiments, 11 out of 21 consecutive residues are positively charged.

In some embodiments, at least 20% of the residues of the non-amphipathic alpha-helix have hydrophobic side chains (e.g., 20% to 40%, 20% to 35%, 20% to 30%, 25% to 40%, 25% to 35%, 25% to 30%, 30% to 40%, or 30% to 35% of the residues of the non-amphipathic alpha-helix have hydrophobic side chains), and 40% to 60% of the residues of the non-amphipathic alpha-helix are positively charged (e.g., 45% to 55%, e.g., about 50% of the residues of the non-amphipathic alpha-helix are positively charged) (e.g., selected from arginine, histidine, and lysine). In some embodiments, at least five out of 21 consecutive residues of the non-amphipathic alpha-helix have hydrophobic side chains, and 11 out of 21 consecutive residues are positively charged. In some embodiments, at least 20% of the residues of the non-amphipathic alpha-helix are selected from alanine, leucine, and methionine (e.g., 20% to 40%, 20% to 35%, 20% to 30%, 25% to 40%, 25% to 35%, 25% to 30%, 30% to 40%, or 30% to 35% of the residues of the non-amphipathic alpha helix are selected from alanine, leucine, and methionine), and 40% to 60% of the residues of the non-amphipathic alpha-helix are positively charged (e.g., 45% to 55%, e.g., about 50% of the residues of the non-amphipathic alpha-helix are positively charged) (e.g., selected from arginine, histidine, and lysine). In some embodiments, at least five out of 21 consecutive residues of the non-amphipathic alpha-helix are selected from alanine, leucine, and methionine, and 11 out of 21 consecutive residues are positively charged.

In some embodiments, the non-amphipathic alpha-helix has a hydrophobic moment (pH) of less than 3.0 (e.g., less than 2.8, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.6, less than 0.5, less than 0.4, or less than 0.3; e.g., from 0.3 to 3.0, from 0.5 to 2.5, from 0.6 to 2.0, from 0.7 to 1.5, or from 0.8 to 1.2; e.g., from 0.3 to 0.5, from 0.5 to 0.75, from 0.75 to 1.0, from 1.0 to 1.5, from 1.5 to 2.0, from 2.0 to 2.5, or from 2.5 to 3.0; e.g., about 0.3, about 0.4, about 0.5, about 0.6, about 0.7, about 0.8, about 0.9, about 1.0, about 1.1., about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In particular instances, the non-amphipathic alpha-helix has a pH of less than 1.0 (e.g., less than 0.9, less than 0.8, less than 0.7, less than 0.6, less than 0.5, less than 0.4, or less than 0.3; e.g., from 0.3 to 1.0, from 0.4 to 1.0, from 0.5 to 1.0, from 0.6 to 1.0, from 0.7 to 1.0, from 0.8 to 1.0, or from 0.9 to 1.0; e.g., from 0.3 to 0.5, from 0.5 to 0.75, or from 0.75 to 1.0; e.g., about 0.3, about 0.4, about 0.5, about 0.6, about 0.7, about 0.8, about 0.9, or about 1.0).

In some instances, the non-amphipathic alpha-helix includes the following formula: [P]—[H]-2[P]—[X]—[P]—[H]-2[X]—[P]—[X]—[P]—[X]-2[H]—[X]-3[P]—[X]—[P](Formula 1), wherein [P] is a positively charged residue, [H] is a hydrophobic residue, and [X] is any residue.

In some instances, the non-amphipathic alpha-helix includes the following formula: [P]—[H]-2[P]—[X]—[P]—[H]—[Y]—[X]—[P]—[Z]—[P]—[X]-2[H]—[Z]-3[P]—[Z]-[P](Formula 2), wherein [P] is a positively charged residue (e.g., a positively charged residue selected from the group consisting of K and R), [H] is a hydrophobic residue (e.g., a hydrophobic residue selected from the group consisting of A, L, and M), and [X] is a positively charged residue or a hydrophobic residue (e.g., a positively charged residue or a hydrophobic residue selected from the group consisting of K, R, A, L, and M) [Y] is a positively charged residue or residue with a polar uncharged side chain, and [Z] is an inert residue (e.g., G).

In some embodiments, the non-amphipathic alpha helix includes an amino acid sequence of KARKKKLNKKGRKMAGRKRGR (SEQ ID NO: 11) or a variant thereof including an amino acid sequence that differs from KARKKKLNKKGRKMAGRKRGR (SEQ ID NO: 11) by no more than 6 amino acids (e.g., no more than 5, no more than 4, no more than 3, no more than 2, or no more than 1 amino acid). In some embodiments, the non-amphipathic alpha helix includes the amino acid sequence of KARKX₁KLX₂X₃KGRX₄MAGRKRGR (SEQ ID NO: 12), wherein X₁, X₂, X₃, and X₄ are each, independently, any amino acid. In some embodiments, X₁, X₂, X₃, and X₄ are each, independently, selected from lysine, alanine, asparagine, arginine, and leucine. In some embodiments, the polypeptide includes the amino acid sequence of KARKAKLRLKGRLMAGRKRGR (SEQ ID NO: 13).

In some embodiments, the non-amphipathic alpha helix includes an amino acid sequence having at least 85%, 90%, 95%, 97%, or 99% sequence identity to KARKKKLNKKGRKMAGRKRGR (SEQ ID NO: 11), KARKKKLNKKGRKMAGRKRGRPK (SEQ ID NO: 14), KARKAKLRLKGRLMAGRKRGRPK (SEQ ID NO: 15), KARKAKLRLKGRLMAGRKRGRP (SEQ ID NO: 16), KARKKKLNKKGRKMAGRKRGRP (SEQ ID NO: 17), KARKAKLRLKARLWARHRARACEA (SEQ ID NO: 18), or KARKAKLRLKGRLWARHRACEA (SEQ ID NO: 19).

In some embodiments, the non-amphipathic alpha helix includes the amino acid sequence of

(SEQ ID NO: 11)
KARKKKLNKKGRKMAGRKRGR,
(SEQ ID NO: 14)
KARKKKLNKKGRKMAGRKRGRPK,
(SEQ ID NO: 15)
KARKAKLRLKGRLMAGRKRGRPK,
(SEQ ID NO: 16)
KARKAKLRLKGRLMAGRKRGRP,
(SEQ ID NO: 17)
KARKKKLNKKGRKMAGRKRGRP,
(SEQ ID NO: 18)
KARKAKLRLKARLWARHRARACEA,
or
(SEQ ID NO: 19)
KARKAKLRLKGRLWARHRACEA.

In some embodiments, the polypeptide includes an amino acid sequence having at least 85%, 90%, 95%, 97%, or 99% sequence identity to KARKKKLNKKGRKMAGRKRGRPKKKRKV (SEQ ID NO: 20), KARKKKLNKKGRKMAGRKRGRPKGGGSPAAKRVKLD (SEQ ID NO: 21), KARKAKLRLKGRLMAGRKRGRPKKSGGVKRKKKP (SEQ ID NO: 22), KARKAKLRLKGRLMAGRKRGRPKGGGSPAAKRVKLD (SEQ ID NO: 23), KARKAKLRLKGRLMAGRKRGRPCGGGSPAAKRVKL (SEQ ID NO: 24), KARKAKLRLKGRLMAGRKRGRPCGGSPAAKRVKL (SEQ ID NO: 25), KARKKKLNKKGRKMAGRKRGRPCGGSPKKKRKV (SEQ ID NO: 26), KARKAKLRLKGRLMAGRKRGRPCGGSPKKKRKV (SEQ ID NO: 27), KARKAKLRLKARLWARHRARACEAPAAKRVKL (SEQ ID NO: 28), KARKAKLRLKGRLWARHRACEAPAAKRVKL (SEQ ID NO: 29), or KARKAKLRLKGRLWARHRACEAPKKKRKV (SEQ ID NO: 30).

In some embodiments, the polypeptide includes the amino acid sequence of

| (SEQ ID NO: 20) | |
|---|---|
| | KARKKKLNKKGRKMAGRKRGRPKKKRKV, |
| (SEQ ID NO: 21) | |
| | KARKKKLNKKGRKMAGRKRGRPKGGGSPAAKRVKLD, |
| (SEQ ID NO: 22) | |
| | KARKAKLRLKGRLMAGRKRGRPKKSGGVKRKKKP, |
| (SEQ ID NO: 23) | |
| | KARKAKLRLKGRLMAGRKRGRPKGGGSPAAKRVKLD, |
| (SEQ ID NO: 24) | |
| | KARKAKLRLKGRLMAGRKRGRPCGGGSPAAKRVKL, |
| (SEQ ID NO: 25) | |
| | KARKAKLRLKGRLMAGRKRGRPCGGSPAAKRVKL, |
| (SEQ ID NO: 26) | |
| | KARKKKLNKKGRKMAGRKRGRPCGGSPKKKRKV, |
| (SEQ ID NO: 27) | |
| | KARKAKLRLKGRLMAGRKRGRPCGGSPKKKRKV, |
| (SEQ ID NO: 28) | |
| | KARKAKLRLKARLWARHRARACEAPAAKRVKL, |
| (SEQ ID NO: 29) | |
| | KARKAKLRLKGRLWARHRACEAPAAKRVKL, |
| | or |
| (SEQ ID NO: 30) | |
| | KARKAKLRLKGRLWARHRACEAPKKKRKV. |

In some embodiments, the alpha helix is an amphipathic alpha helix. In some embodiments, the amphipathic alpha helix includes a RALA motif. In some embodiments, the amphipathic alpha helix includes a plurality of RALA motifs. For example, the amphipathic alpha helix may include two, three, four, five, or more RALA motifs. In some embodiments, the amphipathic alpha helix includes three RALA motifs.

In some embodiments, the polypeptide having a RALA motif includes an amino acid sequence having at least 85%, 90%, 95%, 97%, or 99% sequence identity to WEARLARALARALARHLARALARALRACEA (SEQ ID NO: 9). For example, the polypeptide may include the amino acid sequence of WEARLARALARALARHLARALARALRACEA (SEQ ID NO: 9). In some embodiments, the polypeptide includes an amino acid sequence having at least 85%, 90%, 95%, 97%, or 99% sequence identity to WEARLARALARALARHLARALARALRACEAPKKKRKV (SEQ ID NO: 10). For example, the polypeptide may include the amino acid sequence of

(SEQ ID NO: 10)
WEARLARALARALARHLARALARALRACEAPKKKRKV

In some embodiments, the polypeptide having a RALA motif includes an amino acid sequence having at least 85%, 90%, 95%, 97%, or 99% sequence identity to WEARLARALARALARHLARALARALRACEA (SEQ ID NO: 9) and at least one RALA motif (e.g., at least two RALA motifs or all three RALA motifs) is invariant. In some embodiments, the polypeptide includes an amino acid sequence having at least 85%, 90%, 95%, 97%, or 99% sequence identity to WEARLARALARALARHLARALARALRACEAPKKKRKV (SEQ ID NO: 10) and at least one RALA motif (e.g., at least two RALA motifs or all three RALA motifs) is invariant.

In some embodiments, the amphipathic alpha helix includes a KALA motif. In some embodiments, the amphipathic alpha helix includes a plurality of KALA motifs. For example, the amphipathic alpha helix may include two, three, four, five, or more KALA motifs. In some embodiments, the amphipathic alpha helix includes three KALA motifs (e.g., as in SEQ ID NO: 31 (WEAKLAKALAKALAKHLAKALAKALKACEA)).

In some embodiments, the polypeptide having a KALA motif includes an amino acid sequence having at least 85%, 90%, 95%, 97%, or 99% sequence identity to WEAKLAKALAKALAKHLAKALAKALKACEA (SEQ ID NO: 31). For example, the polypeptide may include the amino acid sequence of WEAKLAKALAKALAKHLAKALAKALKACEA (SEQ ID NO: 31). In some embodiments, the polypeptide includes an amino acid sequence having at least 85%, 90%, 95%, 97%, or 99% sequence identity to WEAKLAKALAKALAKHLAKALAKALKACEAPKKKRKV (SEQ ID NO: 32). For example, the polypeptide may include the amino acid sequence of

(SEQ ID NO: 32)
WEAKLAKALAKALAKHLAKALAKALKACEAPKKKRKV

In some embodiments, the polypeptide having a KALA motif includes an amino acid sequence having at least 85%, 90%, 95%, 97%, or 99% sequence identity to WEAKLAKALAKALAKHLAKALAKALKACEA (SEQ ID NO: 31) and at least one KALA motif (e.g., at least two KALA motifs or all three KALA motifs) is invariant. In some embodiments, the polypeptide includes an amino acid sequence having at least 85%, 90%, 95%, 97%, or 99% sequence identity to WEAKLAKALAKALAKHLAKALAKALKACEAPKKKRKV (SEQ ID NO: 32) and at least one KALA motif (e.g., at least two KALA motifs or all three KALA motifs) is invariant.

### Linkers

In some embodiments, polypeptides may include one or more suitable linkers (e.g., flexible polypeptide linkers or rigid polypeptide linkers). In some embodiments, such linkers may separate two or more motifs (e.g., three, four, or more), e.g., alpha helical motifs. In some embodiments, linkers can be used to separate two more domains from one another. The linker may separate, for example, a DNA condensing polypeptide (e.g., an alpha helix, such as a non-amphipathic alpha helix) and an NLS. In some embodiments, linkers may be formed by adding sequences of small hydrophobic amino acids without rotatory potential (such as glycine) and polar serine residues that confer stability and flexibility. Linkers may be soft and allow the domains of the shuttle agents to move. In some embodiments, prolines may be avoided since they can add significant conformational rigidity.

The linker may be, for example, from two to 20 (e.g., 2 to 8, e.g., 2, 3, 4, 5, 6, 7, 8, 9, 10, 11, 12, 13, 14, 15, 16, 17, 18, 19, or 20, e.g., 4 or 5) amino acid residues in length.

In some embodiments, the linkers may be serine/glycine-rich linkers (e.g., GS, GGS (SEQ ID NO: 33), GGSGGGS (SEQ ID NO: 34), GGSGGGSGGGS (SEQ ID NO: 35), or the like). In some embodiments, the linker may comprise or consist of: -Gn-; —Sn—; -(GnSn)n-; -(GnSn)nGn-; -(GnSn)nSn-; -(GnSn)nGn(GnSn)n-; or -(GnSn)nSn(GnSn)n-, wherein G is the amino acid Gly; S is the amino acid Ser; and n is an integer from 1 to 5. For example, the linker may have or include the amino acid sequence of GGGS (SEQ ID NO: 5), KSGG (SEQ ID NO: 6), CGGGS (SEQ ID NO: 7), or CGGS, (SEQ ID NO: 8).

In some embodiments, the linker is a rigid linker. A rigid linker may substantially constrain the DNA condensing polypeptide (e.g., an alpha helix, e.g., non-amphipathic alpha helix contained therein) and the NLS relative to each other. For example, in some embodiments, the linker [L] is an alpha-helix, which may link the non-amphipathic alpha-helix [C] to the NLS [D]. Thus, where the NLS is an alpha-helix, [C]-[L]-[D] may be a continuous alpha-helix. In some instances, [C]-[L]-[D] is non-amphipathic. In some instances, [C]-[L]-[D] has a hydrophobic moment (pH) of less than 3.0 (e.g., less than 2.8, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.6, less than 0.5, less than 0.4, or less than 0.3; e.g., from 0.3 to 3.0, from 0.5 to 2.5, from 0.6 to 2.0, from 0.7 to 1.5, or from 0.8 to 1.2; e.g., from 0.3 to 0.5, from 0.5 to 0.75, from 0.75 to 1.0, from 1.0 to 1.5, from 1.5 to 2.0, from 2.0 to 2.5, or from 2.5 to 3.0; e.g., about 0.3, about 0.4, about 0.5, about 0.6, about 0.7, about 0.8, about 0.9, about 1.0, about 1.1., about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0). In particular instances, [C]-[L]-[D] has a pH of less than 1.0 (e.g., less than 0.9, less than 0.8, less than 0.7, less than 0.6, less than 0.5, less than 0.4, or less than 0.3; e.g., from 0.3 to 1.0, from 0.4 to 1.0, from 0.5 to 1.0, from 0.6 to 1.0, from 0.7 to 1.0, from 0.8 to 1.0, or from 0.9 to 1.0; e.g., from 0.3 to 0.5, from 0.5 to 0.75, or from 0.75 to 1.0; e.g., about 0.3, about 0.4, about 0.5, about 0.6, about 0.7, about 0.8, about 0.9, or about 1.0). In particular instances, the polypeptide contains no cell penetrating peptide domains.

### Nuclear Localization Sequence (NLS)

The polypeptides described herein may include a nuclear localization sequence. A nuclear localization sequence is an amino acid sequence that serves as a signal mediating transport of a protein from the cytoplasm into the nucleus via nuclear transport. The NLS may contain one or more basic regions (e.g., containing lysine or arginine residues). The NLS may be a monopartite or bipartite NLS. The NLS may contain, for example, two basic clusters, which are separated by a short linker in a bipartite NLS. The NLS may be located, for example, at the C-terminus of the polypeptide. Suitable nuclear localization sequences are known in the art.

In some embodiments, the NLS is seven, eight, or nine amino acid residues in length. The NLS may include, for example, a simian virus 40 (SV40) NLS or a c-MYC NLS. The NLS may include the amino acid sequence PKKKRKV (SEQ ID NO: 1). The NLS may include the amino acid sequence PAAKRVKL (SEQ ID NO: 2). The NLS may include the amino acid sequence of PAAKRVKLD (SEQ ID NO: 3) or VKRKKKP (SEQ ID NO: 4).

### Polynucleotides

The polypeptides describe herein are useful for delivery of a polynucleotide. The polypeptide may be formulated in a composition with a polynucleotide. For example, the polypeptide may contain a DNA condensing polypeptide that condenses the polynucleotide to form a suitable delivery complex. The polypeptide may contain a DNA binding domain that associates with, e.g., binds to, the polynucleotide.

In some embodiments, the polypeptide and the polynucleotide are present at a molar ratio (e.g., in the composition) of from about 100:1 to about 10,000,000:1, e.g., a molar ratio of from about 1,000:1 to about 10,000:1 (e.g., about 1,000:1, 2,000:1, 3,000:1, 4,000:1, 5,000:1, 6,000:1, 7,000:1, 8,000:1, 9,000:1, or 10,000:1).

Polynucleotides of the invention include non-viral nucleic acid vectors (e.g., non-viral DNA vectors or non-viral RNA vectors; e.g., circular DNA vectors and circular RNA vectors). In particular instances, polynucleotides, e.g., nucleic acid vectors (e.g., non-viral nucleic acid vectors) are DNA (e.g., circular DNA (e.g., synthetic circular DNA) or linear DNA (e.g., closed ended DNA or doggybone DNA)) or RNA (e.g., circular RNA).

Some embodiments of the present invention include circular DNA vectors. In some instances, circular DNA vectors useful to carry the therapeutic genes (e.g., therapeutic replacement genes) described herein can be plasmid DNA vectors. In particular instances of the present invention, circular DNA vectors differ from conventional plasmid DNA vectors in that they lack plasmid backbone elements (e.g., bacterial elements such as (i) a bacterial origin of replication and/or (ii) a drug resistance gene). In some embodiments, circular DNA vectors encoding any of the therapeutic genes (e.g., therapeutic replacement genes) described herein lack a recombination site (e.g., synthetic circular DNA vectors produced using a cell-free process). In alternative embodiments, circular DNA vectors described herein include a recombination site (e.g., minicircle DNA vectors, nanoplasmids, and the like).

Circular DNA vectors can persist intracellularly (e.g., in quiescent cells, such as post-mitotic cells) as episomes. Polynucleotide vectors provided herein can be devoid of bacterial plasmid DNA components, such as immunogenic components (e.g., immunogenic bacterial signatures (e.g., CpG motifs)) or components additionally or otherwise associated with reduced persistence (e.g., CpG islands). For example, in some embodiments, the vector contains DNA in which at least 50% (e.g., at least 60%, at least 70%, at least 80%, at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or essentially all) of the DNA lacks one or more elements of bacterial plasmid DNA, such as immunogenic components (e.g., immunogenic bacterial signatures (e.g., CpG motifs)) or components additionally or otherwise associated with reduced persistence (e.g., CpG islands). In some embodiments, at least 50% (e.g., at least 60%, at least 70%, at least 80%, at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or essentially all) of the DNA lacks CpG methylation. In some embodiments, the vector contains DNA in which at least 50% (e.g., at least 60%, at least 70%, at least 80%, at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or essentially all) of the DNA lacks bacterial methylation signatures, such as Dam methylation and Dcm methylation. For example, in some embodiments, the vector contains DNA in which at least 50% (e.g., at least 60%, at least 70%, at least 80%, at least 90%, at least 95%, at least 97%, at least 98%, at least 99%, or essentially all) of the GATC sequences are unmethylated (e.g., by Dam methylase). Additionally, or alternatively, the vector contains DNA in which at least 50% (e.g., at least 60%, at least 70%, at least 80%, at least 90%, at least 95%, at least 97%, at least 98%, at least 99%, or essentially all) of the CCAGG sequences and/or CCTGG sequences are unmethylated (e.g., by Dcm methylase).

In some embodiments of any of the aforementioned polynucleotide vectors, the vector is persistent in vivo (e.g., the circularity and non-bacterial nature (i.e., by in vitro (e.g., cell-free) synthesis) are associated with long-term transcription or expression of a therapeutic gene of the DNA vector). In some embodiments, the persistence of the circular DNA vector is from 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector (e.g., a circular vector produced in bacteria or having one or more bacterial signatures not present in the vector of the invention). In some embodiments, the circular DNA vector of the invention persists for one week to four weeks, from one month to four months, from four months to one year, from one year to five years, from five years to twenty years, or from twenty years to fifty years (e.g., at least one week, at least two weeks, at least one month, at least four months, at least one year, at least two years, at least five years, at least ten years, at least twenty years, at least thirty years, at least forty years, or at least fifty years).

A circular DNA vector may include a promoter operably linked 5′ to a therapeutic gene (e.g., therapeutic replacement gene). A promoter is operably linked to a therapeutic gene (e.g., therapeutic replacement gene) if the promoter is capable of effecting transcription of that therapeutic gene (e.g., therapeutic replacement gene). Promoters that can be used as part of circular DNA vectors include constitutive promoters, inducible promoters, native-promoters, and tissue-specific promoters. Examples of constitutive promoters include a cytomegalovirus (CMV) promoter (optionally with the CMV enhancer), a retroviral Rous sarcoma virus (RSV) LTR promoter (optionally with the RSV enhancer), an SV40 promoter, a dihydrofolate reductase promoter, a p-actin promoter, a phosphoglycerol kinase (PGK) promoter, and an EF1-alpha promoter. In particular embodiments of the invention, the circular DNA vector includes a CMV promoter. In some embodiments, the circular DNA vector includes a CAG promoter.

Alternatively, circular DNA vectors of the invention include inducible promoters. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Examples of inducible promoters regulated by exogenously supplied promoters include zinc-inducible sheep metallothionine (MT) promoters, T7 polymerase promoter systems, ecdysone insect promoters, tetracycline-repressible systems, tetracycline-inducible systems, RU486-inducible systems, and rapamycin-inducible systems. Still other types of inducible promoters which may be useful in this context are those which are regulated by a specific physiological state, e.g., temperature, acute phase, a particular differentiation state of the cell, or in replicating cells only. Inducible promoters and inducible systems are available from a variety of commercial sources.

A circular DNA vector of the invention may also include a polyadenylation sequence 3′ to the self-replicating RNA molecule-encoding sequence. Useful polyadenylation sequences include elongated polyadenylation sequences of greater than 20 nt (e.g., greater than 25 nt, greater that 30 nt, greater than 35 nt, greater than 40 nt, greater than 50 nt, greater than 60 nt, greater than 70 nt, or greater than 80 nt, e.g., from 20 to 100 nt, from 30 to 100 nt, from 40 to 100 nt, from 50 to 100 nt, from 60 to 100 nt, from 70 to 100 nt, from 80 to 100 nt, from 100 to 200 nt, from 200 to 300 nt, or from 300 to 400 nt, or greater).

Circular DNA vectors that lack bacterial elements such as a DNA origin of replication and/or a drug resistance gene can persist in an individual longer than conventional DNA vectors (e.g., plasmids) and longer than naked RNA.

Circular DNA vectors can have various sizes and shapes. A circular DNA vector carrying a therapeutic gene (e.g., therapeutic replacement gene) of the invention can be from 2.5 kb to 20 kb in length (e.g., from 5 kb to 19 kb, from 6 kb to 18 kb, from 7 kb to 16 kb, from 8 kb to 14 kb, or from 9 kb to 12 kb in length, e.g., from 5 kb to 6 kb, from 6 kb to 7 kb, from 7 kb to 8 kb, from 8 kb to 9 kb, from 9 kb to 10 kb, from 10 kb to 11 kb, from 11 kb to 12 kb, from 12 kb to 13 kb, from 13 kb to 14 kb, from 14 kb to 15 kb, from 15 kb to 16 kb, from 16 kb to 18 kb, or from 18 kb to 20 kb in length, e.g., about 3 kb, about 4 kb, about 5 kb, about 6 kb, about 7 kb, about 8 kb, about 9 kb, about 10 kb, about 10.5 kb, about 11 kb, about 11.5 kb, about 12 kb, about 12.5 kb, about 13 kb, about 14 kb, about 15 kb, about 16 kb, about 17 kb, about 18 kb, about 19 kb, or about 20 kb in length).

Circular DNA vectors useful as part of the present invention can be readily synthesized through various means known in the art and described herein. For example, circular DNA vectors that lack plasmid backbone elements (e.g., bacterial elements such as (i) a bacterial origin of replication and/or (ii) a drug resistance gene) can be made using in-vitro (cell-free) methods, which can provide purer compositions relative to bacterial-based methods. Such in-vitro synthesis methods may involve use of phage polymerase, such as Phi29 polymerase, as a replication tool using, e.g., rolling circle amplification. Particular methods of in-vitro synthesis of circular DNA vectors are further described in International Patent Publication WO 2019/178500, which is incorporated herein by reference.

In some instances, the nucleic acid vector is a non-viral nucleic acid vector (e.g., the nucleic acid vector is not encapsulated within a viral capsid).

Polynucleotides described herein may include a therapeutic gene, such as a therapeutic gene or therapeutic sequence encoding a therapeutic replacement protein. A therapeutic replacement protein can replace a protein that is endogenously expressed in a healthy cell or a non-functional mutant protein expressed by the individual being treated. Thus, it will be appreciated that the present polynucleotide vectors encoding therapeutic replacement proteins can be administered as gene replacement therapies and/or gene augmentation therapies.

### Lipids

The compositions described herein may include one or more lipids useful for formulating a polypeptide and a polynucleotide for delivery. Lipid-based structures include a defined complex of lipids held together by noncovalent bonds, such as hydrogen bonds, Van der Waals forces, electrostatic interactions, hydrophobic effect, and Pi-Pi interactions. Lipid-based structures may include large complexes of molecules that form sphere-, rod-, or sheet-like structures. Lipid-based structures include, for example, micelles, liposomes, and lipid nanoparticles (LNPs). Lipid-based structures may have a predetermined size. The size of the structure may vary based on the components (e.g., polynucleotide or polypeptide) packed within the structure.

The Z-average mean particle diameter of the lipid-based structure may vary from, e.g., about 10 nm to about 1000 nm (e.g., from about 10 nm to about 500 nm, or from about 10 nm to about 250 nm). When the structure is an LNP or micelle, the Z-average mean particle diameter may be from about 75 nm to about 250 nm. When the lipid-based structure is a vesicle (e.g., a liposome), the Z-average mean particle diameter may be from about 250 nm to about 750 nm. Non-limiting examples of the Z-average mean particle diameters include, e.g., from about 75 nm to about 100 nm, e.g., from 75 nm to about 85 nm, e.g., about 80 nm, e.g., from about 80 nm to about 140 nm, from about 90 nm to about 130 nm, or from about 110 nm to about 130 nm, e.g., about 120 nm, e.g., from about 200 nm to about 300 nm, e.g., from about 250 nm to about 300 nm, from about 260 nm to about 290 nm, from about 260 nm to about 280 nm, from about 265 nm to about 275 nm, e.g., about 270 nm, e.g., from about 300 nm to about 400 nm, from about 400 nm to about 600 nm, e.g., from about 450 nm to about 550 nm, from about 475 nm to about 525 nm, from about 480 nm to about 520 nm, from about 490 nm to about 510 nm, from about 495 nm to about 505 nm, e.g., about 500 nm, e.g., about 10 nm, about 15 nm, about 20 nm about 25 nm, about 30 nm about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 305 nm, about 310 nm, about 315 nm, about 320 nm, about 325 nm, about 330 nm, about 335 nm, about 340 nm, about 345 nm, about 350 nm, about 355 nm, about 360 nm, about 365 nm, about 370 nm, about 375 nm, about 380 nm, about 385 nm, about 390 nm, about 395 nm, about 400 nm, about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, about 500 nm, about 505 nm, about 510 nm, about 515 nm, about 520 nm, about 525 nm, about 530 nm, about 535 nm, about 540 nm, about 545 nm, about 550 nm, about 555 nm, about 560 nm, about 565 nm, about 570 nm, about 575 nm, about 580 nm, about 585 nm, about 590 nm, about 595 nm, about 600 nm, about 605 nm, about 610 nm, about 615 nm, about 620 nm, about 625 nm, about 630 nm, about 635 nm, about 640 nm, about 645 nm, about 650 nm, about 655 nm, about 660 nm, about 665 nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, about 750 nm, about 755 nm, about 760 nm, about 765 nm, about 770 nm, about 780 nm, about 785 nm, about 780 nm, about 785 nm, about 790 nm, about 795 nm, about 800 nm, about 805 nm, about 810 nm, about 815 nm, about 820 nm, about 825 nm, about 830 nm, about 835 nm, about 840 nm, about 845 nm, about 850 nm, about 855 nm about 860 nm, about 865 nm, about 870 nm, about 875 nm, about 880 nm, about 885 nm, about 890 nm, about 895 nm, about 900 nm, about 905 nm, about 910 nm, about 915 nm, about 920 nm, about 925 nm, about 930 nm, about 935 nm, about 940 nm, about 945 nm, about 950 nm, about 955 nm, about 960 nm, about 965 nm, about 970 nm, about 975 nm, about 980 nm, about 985 nm, about 990 nm, or about 1,000 nm.

In particular embodiments, the structure (e.g., LNP) has a Z-average mean particle diameter from about 10 nm to about 500 nm (e.g., from about 10 nm to about 250 nm, e.g., about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, or 500 nm) as measured by dynamic light scattering (DLS). In some embodiments, the composition contains lipid nanoparticles and at least 90% (e.g., at least 95%, at least 97%, at least 99%, or substantially all) of the nanoparticles in the composition have a diameter from about 10 nm to about 500 nm (e.g., from about 10 nm to about 250 nm, e.g., about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, or 500 nm) as measured by dynamic light scattering (DLS).

The mean particle diameter may be measured by zeta potential, dynamic light scattering (DLS), electrophoretic light scattering (ELS), static light scattering (SLS), molecular weight, electrophoretic mobility, size exclusion chromatography (SEC), field flow fractionation, or other methods known in the art. In some embodiments, the mean particle diameter is measured by DLS. One of skill in the art would appreciate that a population of structures (e.g., liposomes, LNPs, or micelles) may have a range of Z-average mean particle diameters within the population. Thus, the population may be polydisperse. The population may have a polydispersity index of 0.3 or less (e.g., 0.05 to 0.3). The polydispersity index can be determined using DLS (see, e.g., ISO 22412:2017).

The Lipid-based structures may include an endosomal escape moiety. Lipid-based structures including an endosomal escape moiety may provide for an improved cytosolic delivery of the cargo (e.g., a therapeutic agent) included in the structure. Endosomal escape moieties are known in the art. Preferably, an endosomal escape moiety is an ionizable lipid. The ionizable lipids may also serve as structure-layer forming lipids. Non-limiting examples of ionizable lipids include those described in, e.g., WO 2019/067875; WO 2018/191750; and U.S. Pat. No. 9,999,671, which are herein incorporated by reference in their entirety. Other endosomal escape moieties include, for example, fusogenic lipids (e.g., dioleoylphosphatidyl-ethanolamine (DOPE)); and polymers such as polyethylenimine (PEI); poly(beta-amino ester)s; polypeptides, such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Specific examples of endosomal escape moieties including moieties suitable for inclusion in, or conjugation to, to the lipid-based structures disclosed herein are provided, e.g., in WO 2015/188197; the disclosure of these endosomal escape moieties is incorporated by reference herein.

In some embodiments, the lipid-based structures include a lipid that is a phospholipid.

In some embodiments, the lipid is a cationic lipid. In some embodiments, the cationic lipid is 4-(dimethylamino)-butanoic acid, (10Z,13Z)-1-(9Z,12Z)-9,12-octadecadien-1-yl-10,13-nonadecadien-1-yl ester (DLin-MC3-DMA), 1,2-dioleoyl-3-dimethylammonium-propane (DODAP), 1,2-dioleyloxy-3-dimethylaminopropane (DODMA), N,N-dimethyl-2,2-di-(9Z,12Z)-9,12-octadecadien-1-yl-1,3-dioxolane-4-ethanamine (DLin-KC2-DMA), 4-(dimethylamino)-butanoic acid, (10Z,13Z)-1-(9Z,12Z)-9,12-octadecadien-1-yl-10,13-nonadecadien-1-yl ester (DLin-MC3-DMA), OF-02, 3,6-bis({4-[bis(2-hydroxydodecyl)amino]butyl})piperazine-2,5-dione (CKK-E12), 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), or 2-(dioctylamino)ethyl nonyl hydrogen phosphate (9A1P9). OF-02 is described, e.g., in Fenton, et al. Advanced Mater. 28: 2939-2943, 2016, which is herein incorporated by reference in its entirety.

In some embodiments, the lipid is a PEGylated lipid. In some embodiments, the PEGylated lipid is 1,2-Dimyristoyl-sn-glycero-3-methoxypolyethylene glycol (DMG-PEG). For example, the DMP-PEG may be DMG-PEG 2000.

In some embodiments, the lipid is an anionic or neutral lipid. In some embodiments, the lipid is an anionic lipid. In some embodiments, the lipid is a neutral lipid. In some embodiments, the neutral lipid is 1,2-Dioleoyl-sn-glycero-3-phosphoethanolamine (DOPE).

In some embodiments, the lipid is a sterol. The sterol may be, for example, cholesterol or a derivative thereof.

In some embodiments, the lipid-based structure includes a mixture of lipids. For example, in some embodiments, the lipid-based structure includes a cationic lipid, a neutral lipid, an anionic lipid, a PEGylated lipid, a sterol, or any combination or variations thereof. In some embodiments, the lipid-based structure includes a cationic lipid, a neutral lipid, a PEGylated lipid, and a sterol. In some embodiments, the lipid-based structure includes DLin-MC3-DMA, DOPE, cholesterol, DMG-PEG 2000, or a combination thereof. In some embodiments, the lipid-based structure includes a mixture of DLin-MC3-DMA, DOPE, cholesterol, and DMG-PEG 2000. The mixture may include DLin-MC3-DMA, DOPE, cholesterol, and DMG-PEG 2000 at a molar ratio of about 40-60:15-20:25-35:1-2. For example, the mixture may include DLin-MC3-DMA, DOPE, cholesterol, and DMG-PEG 2000 at a molar ratio of about 50:18.5:30:1.5. In some embodiments, a composition as described herein contains a plurality of lipid nanoparticles containing a mixture of lipids including DLin-MC3-DMA, DOPE, cholesterol, and DMG-PEG 2000.

In some embodiments, the lipid-based structure includes a cationic lipid, a neutral lipid, a sterol, and a PEGylated lipid at a molar ratio of about 40-60:15-20:25-35:1-2. For example, the mixture may include a cationic lipid, a neutral lipid, a sterol, and a PEGylated lipid at a molar ratio of about 50:18.5:30:1.5.

In some embodiments, the lipid-based structure includes a cationic lipid (e.g., DLin-MC3-DMA) and a neutral lipid (e.g., DOPE) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the lipid-based structure includes a cationic lipid (e.g., DLin-MC3-DMA) and a sterol (e.g., cholesterol) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the lipid-based structure includes a cationic lipid (e.g., DLin-MC3-DMA) and a PEGylated lipid (e.g., DMG-PEG) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the lipid-based structure includes a neutral lipid (e.g., DOPE) and a sterol (e.g., cholesterol) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the lipid-based structure includes a neutral lipid (e.g., DOPE) and a PEGylated lipid (e.g., DMG-PEG) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

In some embodiments, the lipid-based structure includes a sterol (e.g., cholesterol and a PEGylated lipid (e.g., DMG-PEG) at a molar ratio of from about 100:1 to about 1:100 (e.g., about 100:1, 90:1, 80:1, 70:1, 60:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50: 1:60, 1:70, 1:80, 1:90, or 1:100).

### Liposomes

Liposomes are useful for the transfer and delivery of polynucleotides and polypeptides. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the polypeptide and polynucleotide are delivered into the cell where the polynucleotide can be targeted to the nucleus, e.g., via the NLS on the polypeptide. The composition of the liposome is usually a combination of phospholipids, usually in combination with steroids, such as cholesterol. Other phospholipids or other lipids may also be used. The physical characteristics of liposomes depend on pH, ionic strength, and the presence of divalent cations.

Preferably, a liposome described herein includes a phospholipid, more preferably, a glycerophospholipid, e.g., a phosphatidylserine. A phosphatidylserine is a glycerol molecule having two hydroxyl groups substituted with fatty acid ester moieties and one hydroxyl group substituted with a phosphodiester moiety that is covalently bonded to serine side chain. A typical structure of a phosphatidylserine is RO—CH₂—CH(OR)—CH₂—OP(O)(OH)—OCH2CH(COOH)NH2, or a salt thereof, where each R is independently a fatty acid acyl. Additionally, or alternatively, a liposome described herein may include, e.g., a lysophospholipid, e.g., a lysophosphatidylserine. A lysophosphatidylserine is a phosphatidylserine missing one of its two fatty acid ester moieties. A typical structure of a lysophosphatidylserine is RO—CH₂—CH(OR)—CH₂—OP(O)(OH)—OCH2CH(COOH)NH2, or a salt thereof, where one R is a fatty acid acyl, and the other R is H. Thus, in certain preferred embodiments, a liposome described herein includes RO—CH₂—CH(OR)—CH₂—OP(O)(OH)—OCH2CH(COOH)NH2, or a salt thereof, where each R is H or a fatty acid acyl, provided that at least one R is a fatty acid acyl.

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Cationic liposomes possess the advantage of being able to fuse to the cell membrane. Non-limiting examples of cationic lipids include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yeethylazanediyedidodecan-2-ol (Tech G1), or a mixture thereof. The cationic lipid can include, for example, from about 20 mol % to about 50 mol % or about 40 mol % of the total lipid present in the particle.

Non-cationic liposomes, although not able to fuse as efficiently with the plasma membrane, may also be used for delivery. Anionic liposome compositions generally are formed from dimyristoyl phosphatidylglycerol, while anionic fusogenic liposomes are formed primarily from dioleoyl phosphatidylethanolamine (DOPE). The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, 1,2-dioleoyl-sn-glycero-3-phospho-L-serine (sodium salt, DOPS), or a mixture thereof. The non-cationic lipid can be, for example, from about 5 mol % to about 90 mol %, about 10 mol %, or about 58 mol % if cholesterol is included, of the total lipid present in the particle. In some embodiments, an ionizable/non-cationic lipid can be a combination of lipids described above, e.g., a combination of lipids including DOPC, DOPS, Chol, and DOPE.

The conjugated lipid that inhibits aggregation of liposomal particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C1), a PEG-dimyristyloxypropyl (C14), a PEG-dipalmityloxypropyl (C16), or a PEG-distearyloxypropyl (C18). The conjugated lipid that prevents aggregation of particles can be, for example, from 0 mol % to about 20 mol % or about 2 mol % of the total lipid present in the particle. In some embodiments, the liposome composition further includes cholesterol at, e.g., about 10 mol % to about 60 mol % or about 50 mol % of the total lipid present in the particle.

Another type of liposomal composition is formed from phosphatidylcholine (PC) such as, for example, soybean PC, and egg PC. Another type is formed from mixtures of phospholipid and/or phosphatidylcholine and/or cholesterol. Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Pub. No. 20060058255, the linking groups of which are herein incorporated by reference.

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid; peptidases (which can be substrate specific); and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissues. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate linkers are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

### Lipid Nanoparticles

Polypeptides of in the invention may be encapsulated in a lipid formulation, e.g., a lipid nanoparticle (LNP). LNPs are extremely useful for systemic applications, as they exhibit extended circulation lifetimes following intravenous (i.v.) injection and accumulate at distal sites (e.g., sites physically separated from the administration site).

In some embodiments, the nanoparticles have a diameter from about 10 nm to about 500 nm (e.g., from about 10 nm to about 250 nm, e.g., about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, or 500 nm), e.g., as measured by DLS. The particles of the present invention may have a mean diameter of about 50 nm to about 150 nm, about 60 nm to about 130 nm, about 70 nm to about 110 nm, or about 70 nm to about 90 nm.

In one embodiment, the lipid to drug ratio (mass/mass ratio) (e.g., lipid to oligonucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Non-limiting examples of cationic lipids include DODAC, DDAB, DOTAP, DOTMA, DODMA, DLinDMA, DLenDMA, DLin-C-DAP, DLin-DAC, DLin-MA, DLinDAP, DLin-S-DMA, DLin-2-DMAP, DLin-TMA.CI, DLin-TAP.CI, 1 DLin-MPZ, DLinAP, DOAP, DLin-EG-DMA, (DLin-K-DMA or analogs thereof, ALN100, MC3, Tech G1, or a mixture thereof. The cationic lipid can include, for example, from about 20 mol % to about 50 mol % or about 40 mol % of the total lipid present in the particle.

The ionizable/non-cationic lipid can be an anionic lipid or a neutral lipid including, but not limited to, DSPC, DOPC, DOPS, DPPC, DOPG, DPPG, DOPE, POPC, POPE, DOPE-mal, DPPE, DMPE, DSPE, 16-O-monomethyl PE, 16-O-dimethyl PE, 18-1-trans PE, SOPE, cholesterol, or a mixture thereof.

The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C1), a PEG-dimyristyloxypropyl (C14), a PEG-dipalmityloxypropyl (C16), or a PEG-distearyloxypropyl (C18). The conjugated lipid that prevents aggregation of particles can be, for example, from 0 mol % to about 20 mol % or about 2 mol % of the total lipid present in the particle.

In some embodiments, the LNP further includes cholesterol at, e.g., about 10 mol % to about 60 mol % or about 50 mol % of the total lipid present in the particle.

In some embodiments, the LNP includes a mixture of lipids. For example, in some embodiments, the LNP includes a cationic lipid, a neutral lipid, an anionic lipid, a PEGylated lipid, a sterol, or any combination or variations thereof. In some embodiments, the LNP includes a cationic lipid, a neutral lipid, a PEGylated lipid, and a sterol. In some embodiments, the LNP includes DLin-MC3-DMA, DOPE, cholesterol, DMG-PEG 2000, or a combination thereof. In some embodiments, the LNP includes a mixture of DLin-MC3-DMA, DOPE, cholesterol, and DMG-PEG 2000. The mixture may include DLin-MC3-DMA, DOPE, cholesterol, and DMG-PEG 2000 at a molar ratio of about 40-60:15-20:25-35:1-2. For example, the mixture may include DLin-MC3-DMA, DOPE, cholesterol, and DMG-PEG 2000 at a molar ratio of about 50:18.5:30:1.5. In some embodiments, the LNP contains a plurality of lipid nanoparticles containing a mixture of lipids including DLin-MC3-DMA, DOPE, cholesterol, and DMG-PEG 2000.

### Micelles

Micelles are a particular type of molecular assembly in which amphipathic molecules are arranged in a spherical structure such that all the hydrophobic portions of the molecules are directed inward, leaving the hydrophilic portions in contact with the surrounding aqueous phase. Micelles may be made of lipids. The micelle phase is caused by the packing behavior of single-tail lipids in a bilayer. The difficulty filling all the volume of the interior of a bilayer, while accommodating the area per head group forced on the molecule by the hydration of the lipid head group, leads to the formation of the micelle. This type of micelle is known as a normal-phase micelle (oil-in-water micelle). Inverse micelles have the head groups at the center with the tails extending out (water-in-oil micelle).

Micelles are approximately spherical in shape. Other phases, including shapes such as ellipsoids, cylinders, and bilayers, are also possible. The shape and size of a micelle are a function of the molecular geometry of its surfactant molecules and solution conditions such as surfactant concentration, temperature, pH, and ionic strength. The process of forming micelles is known as micellization and forms part of the phase behavior of many lipids according to their polymorphism.

### Pharmaceutical Compositions and Methods of Use

The polypeptides, polynucleotides, and/or lipids described herein may be formulated into pharmaceutical compositions for administration to human subjects in a biologically compatible form suitable for administration in vivo. Alternatively, the compositions can be administered to a cell in vitro. For example, the composition may be administered for the treatment of a disease or condition requiring administration of a therapeutic protein or nucleic acid.

In some instances, a pharmaceutical composition of the invention includes any of the polypeptides described herein in association with a polynucleotide, which disassociate upon or after entry into a target cell (e.g., entry into the cell or entry into the nucleus of the cell) to facilitate expression of the polynucleotide by the target cell. In some instances, association of the polypeptides with polynucleotides can reduce adverse effects (e.g., immunogenicity) of certain DNA vector compositions (e.g., as compared to the DNA vector composition without the polypeptide). Without being bound by theory, association of polypeptides with polynucleotides can, in some instances, shield the DNA vector from engaging cytosolic signaling pathways (e.g., innate immune pathways), such as cyclic guanosine monophosphate-adenosine monophosphate synthase (cGAS) and/or stimulation of IFN genes (STING).

A pharmaceutical composition having a composition of the invention may contain a pharmaceutically acceptable carrier. Pharmaceutically acceptable carriers in which a composition may be formulated include excipients and/or stabilizers that are nontoxic to the individual at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

If the composition is provided in liquid form, the carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl₂, Cal₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂) or potassium chloride (KCl), wherein further anions may be present. CaCl₂) can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂)). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from theobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

The compositions described herein may be administered to a subject in a variety of forms depending on the selected route of administration, as will be understood by those skilled in the art. The compositions described herein may be administered, for example, by any route that allows the polynucleotide, or composition thereof (e.g., nanoparticle, liposome, micelle, or LNP), to reach the target cell. The composition can be delivered intravenously, subcutaneously, intradermally, percutaneously, intramuscularly, intraarterially, intraperitoneally, intralesionally, intracranially, intraarticularly, intraprostatically, intrapleurally, intratracheally, intrathecally, intranasally, intravaginally, intrarectally, topically, peritoneally, subconjunctivally, intravesicularly, mucosally, intrapericardially, intraumbilically, orally, topically, transdermally, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. The compositions utilized in the methods described herein can be administered systemically. The method of administration can vary depending on various factors (e.g., the compound or composition being administered and the severity of the condition, disease, or disorder being treated).

In one embodiment, the composition is formulated for intraocular administration. For example, the composition may be administered via subretinal injection. In other embodiments, the composition is ocularly administered by non-subretinal injection, such as intravitreal, periocular or suprachoroidal administration. In one embodiment, the composition is administered via parenteral administration. Parenteral administration may be by continuous infusion over a selected period of time. In some preferred embodiments, the compositions described herein are administered via inhalation.

Compositions described herein may be administered, e.g., by inhalation. Inhalation may be oral inhalation or nasal inhalation. An inhalable composition described herein may be provided as a liquid dosage form or dry powder dosage form. A dry powder composition may be, e.g., administered by inhalation as is or after reconstitution in a vehicle (e.g., saline (e.g., isotonic saline), phosphate-buffered saline, or water).

Compositions described herein can be delivered into cells via in vivo electrotransfer (e.g., in vivo electroporation). In vivo electroporation has been demonstrated in certain tissues, such as skin, skeletal muscle, certain tumor types, and lung epithelium. Delivery of polynucleotides into cells by in vivo electroporation involves administration of the polynucleotides into target tissue, followed by application of electrical field to temporarily increase cell membrane permeability within the tissue by generating pores, allowing the polynucleotides to cross cell membranes. As an example, delivery to skin using in vivo electroporation is described in Cha & Daud Hum. Vaccin. Immunother. 2012, 8(11):1734-1738, which is incorporated by reference in its entirety. In vivo electroporation of skeletal muscle is described in Sokolowska & Blachnio-Zabielska, Int. J. Molecular Sci. 2019, 20:2776, which is incorporated by reference in its entirety. Intratumoral delivery using in vivo electroporation is described in Aung et al. Gene Therapy 2009, 16:830-839, which is incorporated by reference in its entirety. In vivo electroporation of DNA into lung cells is described in Pringle et al. J. Gene Med. 2007, 9:369-380, which is incorporated by reference in its entirety. In vivo electrotransfer of polynucleotides to cells in the eye (e.g., retinal cells and/or photoreceptor cells) is described in PCT/US2022/021209, which is incorporated by reference in its entirety. In some instances, after administration of the polynucleotides to the eye (e.g., by suprachoroidal injection, intravitreal injection, or subretinal injection), an electrode can be positioned within the interior of the eye (e.g., within about 1 mm from the retina), and an electric field can be transmitted through the electrode into a target ocular tissue at conditions suitable for electrotransfer of the polynucleotide into the target cell (e.g., by applying six to ten pulses from 10-100 V each). Devices and systems having electrodes suitable for transmitting electric fields in mammalian tissues are commercially available and can be useful in the methods disclosed herein. In some instances, the electric field is transmitted through an electrode comprising a needle (e.g., a needle positioned within the vitreous humor or in the subretinal space). Suitable needle electrodes include CLINIPORATOR® electrodes marketed by IGEA® and needle electrodes marketed by AMBU®. Methods of the invention include administration of any of the polynucleotides described herein, or pharmaceutical compositions thereof, to skin, skeletal muscle, tumors (including, e.g., melanomas), eye, and lung via in vivo electrotransfer.

A composition described herein may also be administered parenterally. Solutions of a composition described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions suitable for buccal or sublingual administration include tablets, lozenges, and pastilles, where the active ingredient is formulated with a carrier, such as sugar, acacia, tragacanth, gelatin, and glycerin. Compositions for rectal administration are conveniently in the form of suppositories containing a conventional suppository base, such as cocoa butter.

The composition described herein may be administered to an animal, e.g., a human, alone or in combination with pharmaceutically acceptable carriers, as noted herein, the proportion of which is determined by the solubility and chemical nature of the composition, chosen route of administration, and standard pharmaceutical practice.

The dosage of the compositions (e.g., a composition including a polypeptide and polynucleotide) described herein, can vary depending on many factors, such as the pharmacodynamic properties of the components of the composition, the mode of administration, the age, health, and weight of the recipient, the nature and extent of the symptoms, the frequency of the treatment, and the type of concurrent treatment, if any, and the clearance rate of the composition in the animal to be treated. The compositions described herein may be administered initially in a suitable dosage that may be adjusted as required, depending on the clinical response. In some embodiments, the dosage of a composition (e.g., a composition including a polypeptide and a polynucleotide) is a prophylactically or a therapeutically effective amount. Furthermore, it is understood that all dosages may be continuously given or divided into dosages given per a given time frame. The composition can be administered, for example, every hour, day, week, month, or year. In some embodiments, the composition may be administered continuously or systemically.

The compositions described herein may be used for introducing a polynucleotide into a target cell by contacting the target cell with a composition as described herein. The target cell may be, for example, a mammalian cell (e.g., a human cell). The contacting may be performed by in vivo administration of the composition to a subject that has the target cell. Alternatively, the contacting may be performed in vitro. Following the in vitro contacting, the target cell may be introduced into the subject. In some embodiments, the polynucleotide is expressed by the target cell. The expression may be measured by detecting a protein encoded by the polynucleotide. Protein expression can be determined by measuring the concentration or relative abundance of a corresponding protein product encoded by a polynucleotide. Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

### EXAMPLES

The following examples provide a description of how the compositions and methods described herein may be used, made, and evaluated, and are intended to be purely exemplary of the disclosure and are not intended to limit the scope of what the inventors regard as their disclosure.

### Example 1. Production of Lipid Nanoparticles

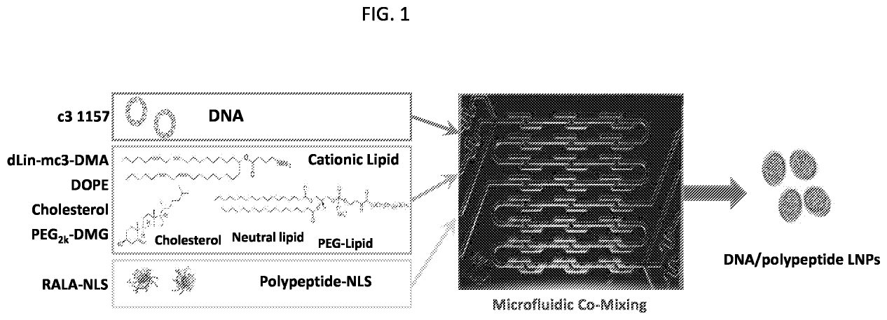

Lipid nanoparticles containing DNA and NLS-containing polypeptides were produced as shown in FIG. 1. The composition included a covalently closed circular (C3) DNA vector polynucleotide encoding luciferase (fLuc1157) (C31157), a cationic lipid (DLin-MC3-DMA), a neutral lipid (DOPE), a PEGylated lipid (DMG-PEG2000), cholesterol, and a RALA polypeptide with a nuclear localization sequence. The components were comixed at a predetermined ratio via independent channels in a microfluidic device to form lipid nanoparticles.

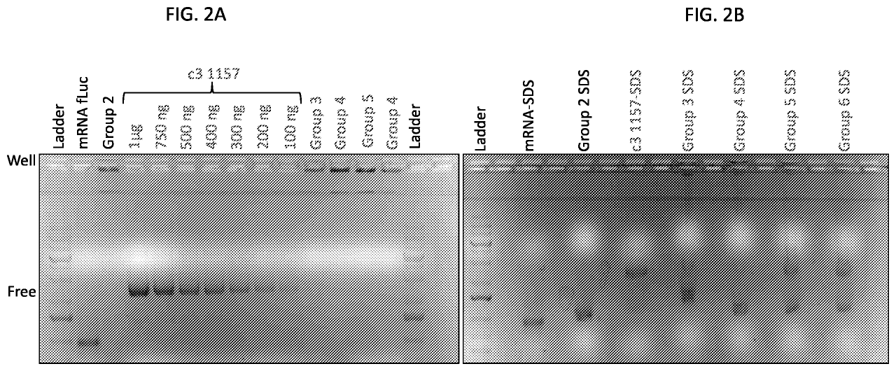

DNA encapsulation was confirmed by gel shift assays. FIGS. 2A and 2B show gel shift assays with various amounts of C³-fLuc1157 DNA encapsulated in LNPs. FIG. 2A shows DNA encapsulated in LNPs; Group 3: LNP containing C³-fLuc1157; Group 4: LNP containing C³-fLuc1157 and RALA-NLS at 1150× molar ratio to C³ DNA. The lack of free DNA in Groups 3 and 4 and DNA retained in the wells shows complete encapsulation in the LNPs. FIG. 2B shows an assessment for nucleic acid integrity and concentration as confirmed by payload release with SDS.

### Example 2. In Vitro Transfection of HepG2 Cells

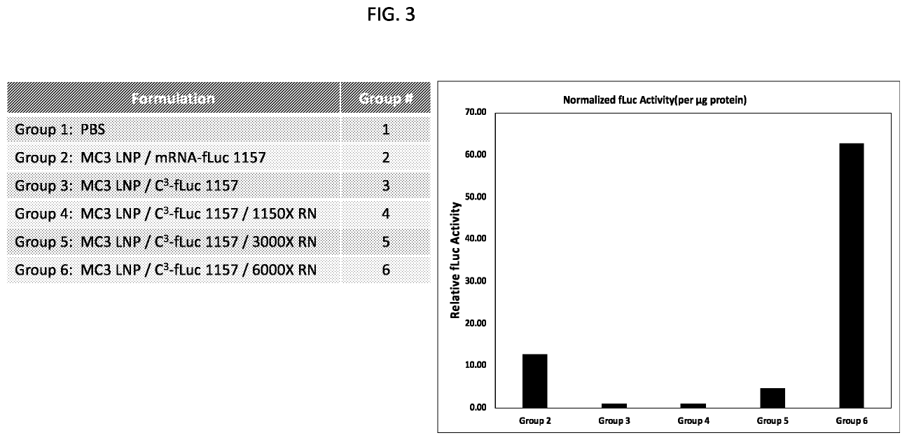

FIG. 3 is a graph showing normalized luciferase activity in HepG2 cells 48 hours after in vitro transfection. Lipid nanoparticles containing fLuc1157 in the presence or absence of RALA-NLS polypeptides at various concentrations were used to transfect HepG2 cells in vitro. Group 1: PBS; Group 2: LNP containing mRNA-fLuc1157; Group 3: LNP containing C³ DNA encoding fLuc1157; Group 4: LNP containing C³-fLuc1157 and RALA-NLS at 1150× molar ratio to C³ DNA; Group 5: LNP containing C3-fLuc1157 and RALA-NLS at 3000× molar ratio to C³ DNA; Group 6: LNP containing C³-fLuc1157 and RALA-NLS at 6000× molar ratio to C³ DNA. As shown in the right part of the graph, Group 6, which contained the highest ratio (6000×) of polypeptide to DNA showed the highest normalized luciferase activity.

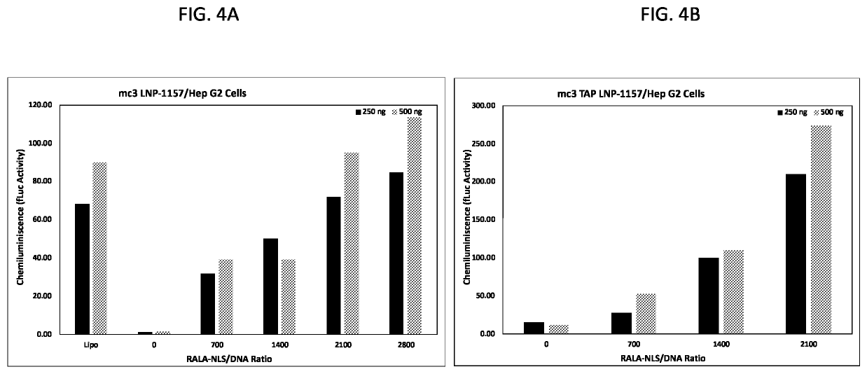

FIGS. 4A and 4B are graphs showing normalized luciferase activity in HepG2 cells 48 hours after in vitro transfection. Lipid nanoparticles were formed with RALA-NLS polypeptides and 250 ng or 500 ng C³-fLuc1157 DNA at a molar ratio of 700, 1400, 2100, or 2800. FIG. 4A shows LNPs containing DOPE, and FIG. 4B shows LNPs containing 20% DOTAP. The bars on the right indicate that higher amounts of DNA (500 ng) produced more efficient transfection as compared to lower amounts (250 ng). LNP formulations with 700, 1400, 2100 and 2800× polypeptide showed transfection improvement by ˜20, 35, 50 and 65-fold respectively, compared to LNP without polypeptide. The transfection efficiency was more pronounced in slightly cationic LNPs (20% DOTAP) as shown in FIG. 4B.

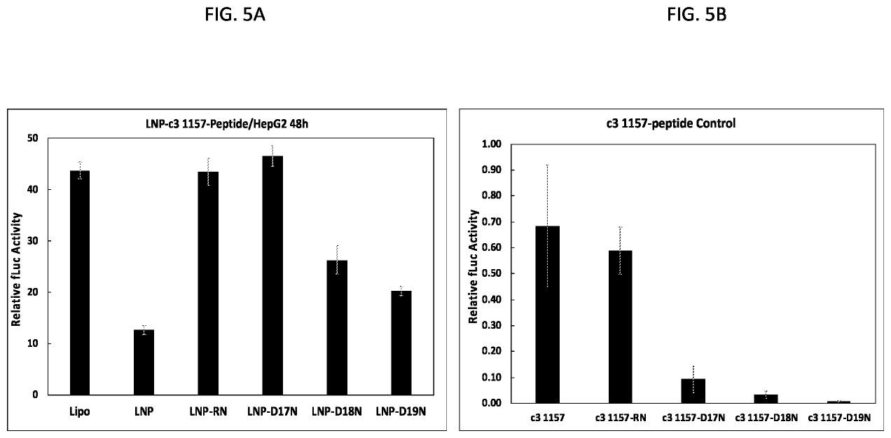

FIGS. 5A and 5B are graphs showing normalized luciferase activity in HepG2 cells 48 hours after in vitro transfection. Lipid nanoparticles were formed with RALA-NLS (RN) polypeptides (WEARLARALARALARHLARALARALRACEAPKKKRKV; SEQ ID NO: 10), DBD17-NLS (KARKAKLRLKARLWARHRARACEAPAAKRVKL; SEQ ID NO: 28), DBD18-NLS (KARKAKLRLKGRLWARHRACEAPAAKRVKL; SEQ ID NO: 29), or DBD19-NLS (KARKAKLRLKGRLWARHRACEAPKKKRKV; SEQ ID NO: 30). FIG. 5A shows LNPs and FIG. 5B shows polypeptide controls without LNP formulation. These experiments show that various constructs produced robust luciferase expression as compared to polypeptide controls.

### Example 3. In Vivo Delivery of C³ DNA to Liver Cells

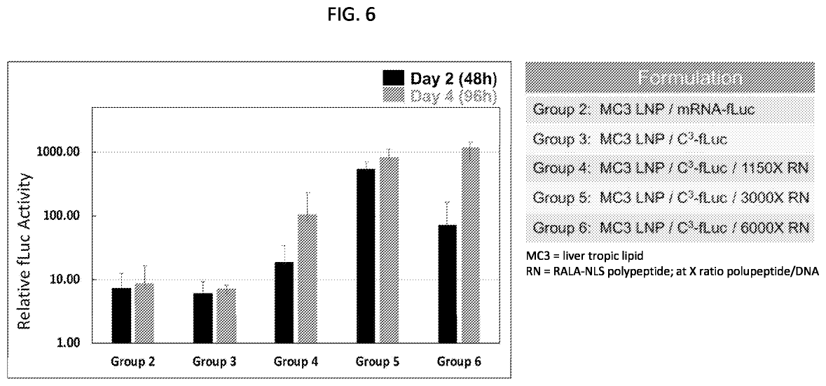

LNP delivery to mouse liver cells was assessed. FIG. 6 shows normalized liver luciferase activity 48 hours and 96 hours after intravenous injection in mouse tail veins. Lipid nanoparticles containing fLuc1157 in the presence or absence of RALA-NLS polypeptides (RALA-NLS) at various concentrations were used to transfect HepG2 cells in vivo. Group 1: PBS; Group 2: LNP containing mRNA-fLuc1157; Group 3: LNP containing C³-fLuc1157; Group 4: LNP containing C³-fLuc1157 and RALA-NLS at 1150× molar ratio to C³ DNA; Group 5: LNP containing C³-fLuc1157 and RALA-NLS at 3000× molar ratio to C3 DNA; Group 6: LNP containing C³-fLuc1157 and RALA-NLS at 6000× molar ratio to C³ DNA. These experiments show robust LNP mediated delivery of C³ DNA to liver cells. mRNA-LNP (Group 2) shows low expression, as mRNA expression peaks within hours of IV dosing. Increase polypeptide:DNA ratios leads to increased fLuc expression levels, correlating with the in vitro results discussed above.

### ORDERED EMBODIMENTS

The following sections describe various embodiments of the invention.

1. A composition comprising:
(a) a polypeptide comprising [A]-[B], wherein:
   [A] is a DNA condensing polypeptide; and
   [B] is a nuclear localization sequence (NLS);
(b) a polynucleotide; and
(c) a lipid.

2. The composition of embodiment 1, wherein the polypeptide is from 2 kDa to 5 kDa.

3. The composition of embodiment 1 or 2, wherein the polypeptide is from 20 to 50 amino acid residues in length.

4. The composition of any one of embodiments 1-3, wherein 30-50% of the residues of the polypeptide are cationic.

5. The composition of any one of embodiments 1-4, wherein the NLS is seven, eight, or nine amino acid residues in length.

6. The composition of any one of embodiments 1-5, wherein the NLS comprises the amino acid sequence PKKKRKV (SEQ ID NO: 1).

7. The composition of any one of embodiments 1-5, wherein the NLS comprises the amino acid sequence PAAKRVKL (SEQ ID NO: 2).

8. The composition of any one of embodiments 1-5, wherein the NLS comprises the amino acid sequence of PAAKRVKLD (SEQ ID NO: 3) or VKRKKKP (SEQ ID NO: 4).

9. The composition of any one of embodiments 1-6, wherein the polypeptide comprises a linker between [A] and [B].

10. The composition of embodiment 9, wherein the linker is from two to 20 amino acid residues in length.

11. The composition of embodiment 10, wherein the linker is from two to eight amino acid residues in length.

12. The composition of embodiment 11, wherein the linker is four or five amino acid residues in length.

13. The composition of any one of embodiments 9-12, wherein the linker comprises one or more glycines or serines.

14. The composition of any one of embodiments 9-13, wherein the linker comprises the amino acid sequence of GGGS (SEQ ID NO: 5), KSGG (SEQ ID NO: 6), CGGGS (SEQ ID NO: 7), or CGGS, (SEQ ID NO: 8).

15. The composition of any one of embodiments 9-12, wherein the linker is a rigid linker.

16. The composition of any one of embodiments 1-15, wherein the DNA condensing polypeptide comprises an amphipathic alpha helix.

17. The composition of any one of embodiment 16, wherein the amphipathic alpha helix is from 12 to 42 amino acid residues in length.

18. The composition of embodiment 17, wherein the amphipathic alpha helix is from 20 to 30 amino acid residues in length.

19. The composition of any one of embodiments 16-18, wherein the amphipathic alpha helix comprises a RALA motif.

20. The composition of embodiment 19, wherein the amphipathic alpha helix comprises a plurality of RALA motifs.

21. The composition of embodiment 20, wherein the amphipathic alpha helix comprises three RALA motifs.

22. The composition of any one of embodiments 1-21, wherein the DNA condensing polypeptide comprises an amino acid sequence having at least 85%, 90%, 95%, 97%, or 99% sequence identity to WEARLARALARALARHLARALARALRACEA (SEQ ID NO: 9).

23. The composition of embodiment 22, wherein the DNA condensing polypeptide comprises the amino acid sequence of WEARLARALARALARHLARALARALRACEA (SEQ ID NO: 9).

24. The composition of embodiment 22, wherein the polypeptide comprises an amino acid sequence having at least 85%, 90%, 95%, 97%, or 99% sequence identity to

(SEQ ID NO: 10)
WEARLARALARALARHLARALARALRACEAPKKKRKV

25. The composition of embodiment 24, wherein the polypeptide comprises the amino acid sequence of WEARLARALARALARHLARALARALRACEAPKKKRKV (SEQ ID NO: 10).

26. The composition of any one of embodiments 1-15, wherein the DNA condensing polypeptide comprises a DNA binding domain.

27. The composition of embodiment 26, wherein the DNA binding domain comprises an alpha helix.

28. The composition of embodiment 27, wherein the alpha helix is from 12 to 42 amino acid residues in length.

29. The composition of embodiment 28, wherein the alpha helix is from 20 to 30 amino acid residues in length.

30. The composition of any one of embodiments 27-29, wherein the alpha helix is a non-amphipathic alpha helix.

31. The composition of embodiment 30, wherein the non-amphipathic alpha helix has a hydrophobic moment (pH) of less than 1.0.

32. The composition of embodiment 30 or 31, wherein the non-amphipathic alpha helix comprises an amino acid sequence of KARKKKLNKKGRKMAGRKRGR (SEQ ID NO: 11) or a variant thereof comprising an amino acid sequence that differs from KARKKKLNKKGRKMAGRKRGR (SEQ ID NO: 11) by no more than 6 amino acids.

33. The composition of embodiment 32, wherein the non-amphipathic alpha helix comprises the amino acid sequence of KARKX₁KLX₂X₃KGRX₄MAGRKRGR (SEQ ID NO: 12), wherein X₁, X₂, X₃, and X₄ are each, independently, any amino acid.

34. The composition of embodiment 33, wherein X₁, X₂, X₃, and X₄ are each, independently, selected from lysine, alanine, asparagine, arginine, and leucine.

35. The composition of embodiment 34, wherein the non-amphipathic alpha helix comprises the amino acid sequence of KARKAKLRLKGRLMAGRKRGR (SEQ ID NO: 13).

36. The composition of embodiment 30 or 31, wherein the non-amphipathic alpha helix comprises an amino acid sequence having at least 85%, 90%, 95%, 97%, or 99% sequence identity to

| (SEQ ID NO: 11) | |
|---|---|
| | KARKKKLNKKGRKMAGRKRGR, |
| (SEQ ID NO: 14) | |
| | KARKKKLNKKGRKMAGRKRGRPK, |
| (SEQ ID NO: 15) | |
| | KARKAKLRLKGRLMAGRKRGRPK, |
| (SEQ ID NO: 16) | |
| | KARKAKLRLKGRLMAGRKRGRP, |
| (SEQ ID NO: 17) | |
| | KARKKKLNKKGRKMAGRKRGRP, |
| (SEQ ID NO: 18) | |
| | KARKAKLRLKARLWARHRARACEA, |
| | or |
| (SEQ ID NO: 19) | |
| | KARKAKLRLKGRLWARHRACEA. |

37. The composition of embodiment 36, wherein the non-amphipathic alpha helix comprises the amino acid sequence of KARKKKLNKKGRKMAGRKRGR (SEQ ID NO: 11), KARKKKLNKKGRKMAGRKRGRPK (SEQ ID NO: 14), KARKAKLRLKGRLMAGRKRGRPK (SEQ ID NO: 15), KARKAKLRLKGRLMAGRKRGRP (SEQ ID NO: 16), KARKKKLNKKGRKMAGRKRGRP (SEQ ID NO: 17), KARKAKLRLKARLWARHRARACEA (SEQ ID NO: 18), or KARKAKLRLKGRLWARHRACEA (SEQ ID NO: 19).

38. The composition of embodiment 36, wherein the polypeptide comprises an amino acid sequence having at least 85%, 90%, 95%, 97%, or 99% sequence identity to

| (SEQ ID NO: 20) | |
|---|---|
| | KARKKKLNKKGRKMAGRKRGRPKKKRKV, |
| (SEQ ID NO: 21) | |
| | KARKKKLNKKGRKMAGRKRGRPKGGGSPAAKRVKLD, |
| (SEQ ID NO: 22) | |
| | KARKAKLRLKGRLMAGRKRGRPKKSGGVKRKKKP, |
| (SEQ ID NO: 23) | |
| | KARKAKLRLKGRLMAGRKRGRPKGGGSPAAKRVKLD, |
| (SEQ ID NO: 24) | |
| | KARKAKLRLKGRLMAGRKRGRPCGGGSPAAKRVKL, |
| (SEQ ID NO: 25) | |
| | KARKAKLRLKGRLMAGRKRGRPCGGSPAAKRVKL, |
| (SEQ ID NO: 26) | |
| | KARKKKLNKKGRKMAGRKRGRPCGGSPKKKRKV, |
| (SEQ ID NO: 27) | |
| | KARKAKLRLKGRLMAGRKRGRPCGGSPKKKRKV, |
| (SEQ ID NO: 28) | |
| | KARKAKLRLKARLWARHRARACEAPAAKRVKL, |
| (SEQ ID NO: 29) | |
| | KARKAKLRLKGRLWARHRACEAPAAKRVKL, |
| | or |
| (SEQ ID NO: 30). | |
| | KARKAKLRLKGRLWARHRACEAPKKKRKV |

39. The composition of embodiment 38, wherein the polypeptide comprises the amino acid sequence of KARKKKLNKKGRKMAGRKRGRPKKKRKV (SEQ ID NO: 20), KARKKKLNKKGRKMAGRKRGRPKGGGSPAAKRVKLD (SEQ ID NO: 21), KARKAKLRLKGRLMAGRKRGRPKKSGGVKRKKKP (SEQ ID NO: 22), KARKAKLRLKGRLMAGRKRGRPKGGGSPAAKRVKLD (SEQ ID NO: 23), KARKAKLRLKGRLMAGRKRGRPCGGGSPAAKRVKL (SEQ ID NO: 24), KARKAKLRLKGRLMAGRKRGRPCGGSPAAKRVKL (SEQ ID NO: 25), KARKKKLNKKGRKMAGRKRGRPCGGSPKKKRKV (SEQ ID NO: 26), KARKAKLRLKGRLMAGRKRGRPCGGSPKKKRKV (SEQ ID NO: 27), KARKAKLRLKARLWARHRARACEAPAAKRVKL (SEQ ID NO: 28), KARKAKLRLKGRLWARHRACEAPAAKRVKL (SEQ ID NO: 29), or KARKAKLRLKGRLWARHRACEAPKKKRKV (SEQ ID NO: 30).

40. The composition of any one of embodiments 1-39, wherein the polynucleotide is from about 500 nucleotides to about 20,000 nucleotides in length.

41. The composition of any one of embodiments 1-40, wherein the polynucleotide encodes a protein.

42. The composition of any one of embodiments 1-41, wherein the polynucleotide is a non-viral polynucleotide.

43. The composition of any one of embodiments 1-42, wherein the polynucleotide is a closed circular polynucleotide.

44. The composition of any one of embodiment 1-43, wherein the polynucleotide is DNA.

45. The composition of embodiment 43 or 44, wherein the polynucleotide is closed circular supercoiled DNA.

46. The composition of any one of embodiments 1-45, wherein the polypeptide and the polynucleotide are present at a molar ratio of from about 100:1 to about 10,000,000:1.

47. The composition of embodiment 46, wherein the polypeptide and the polynucleotide are present at a molar ratio of from about 1,000:1 to about 10,000:1.

48. The composition of any one of embodiments 1-47, wherein the lipid is a phospholipid.

49. The composition of any one of embodiments 1-48, wherein the lipid is a cationic lipid.

50. The composition of embodiment 49, wherein the cationic lipid is 4-(dimethylamino)-butanoic acid, (10Z,13Z)-1-(9Z,12Z)-9,12-octadecadien-1-yl-10,13-nonadecadien-1-yl ester (DLin-MC3-DMA), 1,2-dioleoyl-3-dimethylammonium-propane (DODAP), 1,2-dioleyloxy-3-dimethylaminopropane (DODMA), N,N-dimethyl-2,2-di-(9Z,12Z)-9,12-octadecadien-1-yl-1,3-dioxolane-4-ethanamine (DLin-KC2-DMA), 4-(dimethylamino)-butanoic acid, (10Z,13Z)-1-(9Z,12Z)-9,12-octadecadien-1-yl-10,13-nonadecadien-1-yl ester (DLin-MC3-DMA), OF-02, 3,6-bis({4-[bis(2-hydroxydodecyl)amino]butyl})piperazine-2,5-dione (CKK-E12), 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), or 2-(dioctylamino)ethyl nonyl hydrogen phosphate (9A1P9).

51. The composition of any one of embodiments 1-50, wherein the lipid is a PEGylated lipid.

52. The composition of embodiment 51, wherein the PEGylated lipid is 1,2-Dimyristoyl-sn-glycero-3-methoxypolyethylene glycol (DMG-PEG).

53. The composition of embodiment 52, wherein the DMG-PEG is DMG-PEG 2000.

54. The composition of any one of embodiments 1-47, wherein the lipid is an anionic or neutral lipid.

55. The composition of embodiment 49, wherein the neutral lipid is 1,2-Dioleoyl-sn-glycero-3-phosphoethanolamine (DOPE).

56. The composition of any one of embodiments 1-47, wherein the lipid is a sterol.

57. The composition of embodiment 56, wherein the sterol is cholesterol or a derivative thereof.

58. The composition of any one of embodiments 1-57, wherein the composition comprises a mixture of lipids.

59. The composition of any one of embodiments 1-58, wherein the composition comprises DLin-MC3-DMA, DOPE, cholesterol, DMG-PEG 2000, or a combination thereof.

60. The composition of embodiment 59, wherein the composition comprises a mixture of DLin-MC3-DMA, DOPE, cholesterol, and DMG-PEG 2000.

61. The composition of embodiment 60, wherein the mixture comprises DLin-MC3-DMA, DOPE, cholesterol, and DMG-PEG 2000 at a molar ratio of about 40-60:15-20:25-35:1-2.

62. The composition of embodiment 61, wherein the mixture comprises DLin-MC3-DMA, DOPE, cholesterol, and DMG-PEG 2000 at a molar ratio of about 50:18.5:30:1.5.

63. The composition of any one of embodiments 1-62, wherein the composition comprises the polypeptide and the polynucleotide at a molar ratio of from about 1:1 to about 10,000:1.

64. The composition of embodiment 63, wherein the composition comprises the polypeptide and the polynucleotide at a molar ratio of from about 1000:1 to about 7,000:1.

65. The composition of embodiment 64, wherein the composition comprises the polypeptide and the polynucleotide at a molar ratio of about 1500:1, about 3000:1, or about 6000:1.

66. The composition of any one of embodiments 1-65, wherein the composition comprises a nanoparticle comprising the polypeptide, the polynucleotide, and the lipid.

67. The composition of any one of embodiments 1-66, wherein the composition comprises a plurality of the polypeptides, a plurality of the polynucleotides, and a plurality of the lipids.

68. The composition of embodiment 67, wherein the comprises a plurality of nanoparticles.

69. The composition of embodiment 68, wherein at least 90% of the nanoparticles in the composition have a diameter from about 10 nm to about 500 nm as measured by dynamic light scattering (DLS).

70. The composition of embodiment 69, wherein at least 90% of the nanoparticles in the composition have a diameter from about 10 nm to about 250 nm as measured by DLS.

71. A method of introducing a polynucleotide into a target cell comprising contacting the target cell with the composition of any one of embodiments 1-70.

72. The method of embodiment 71, wherein the target cell is a mammalian cell.

73. The method of embodiment 72, wherein the mammalian cell is a human cell.

74. The method of any one of embodiments 71-73, wherein the contacting is performed by in vivo administration of the composition to a subject comprising the target cell.

75. The method of any one of embodiments 71-74, wherein the contacting with the target cell is performed in vitro.

76. The method of embodiment 75, wherein the target cell is introduced into the subject following the in vitro contacting.

77. The method of any one of embodiments 71-76, wherein the polynucleotide is expressed by the target cell.

78. The method of embodiment 77, wherein the expression is measured by detecting a protein encoded by the polynucleotide.

79. A polypeptide comprising [C]-[L]-[D], wherein:
[C] is a non-amphipathic alpha helix from 12 to 42 amino acid residues in length;
[L] is a linker from one to 20 amino acid residues in length or is absent; and
[D] is a nuclear localization sequence (NLS) from five to 12 amino acid residues in length.

80. The polypeptide of embodiment 79, wherein the linker is from two to eight amino acid residues in length.

81. The polypeptide of embodiment 80, wherein the linker is four or five amino acid residues in length.

82. The polypeptide of any one of embodiments 79-81, wherein the linker comprises one or more glycines or serines.

83. The polypeptide of any one of embodiments 79-82, wherein the linker comprises the amino acid sequence of GGGS (SEQ ID NO: 5), KSGG (SEQ ID NO: 6), CGGGS (SEQ ID NO: 7), or CGGS, (SEQ ID NO: 8).

84. The polypeptide of any one of embodiments 79-81, wherein the linker is a rigid linker.

85. The polypeptide of any one of embodiments 79-84, wherein the NLS is seven, eight, or nine amino acid residues in length.

86. The polypeptide of any one of embodiments 79-85, wherein the NLS comprises the amino acid sequence PKKKRKV (SEQ ID NO: 1).

87. The polypeptide of any one of embodiments 79-85, wherein the NLS the amino acid sequence PAAKRVKL (SEQ ID NO: 2).

88. The polypeptide of any one of embodiments 79-85, wherein the NLS comprises the amino acid sequence of PAAKRVKLD (SEQ ID NO: 3) or VKRKKKP (SEQ ID NO: 4).

89. The polypeptide of any one of embodiments 79-88, wherein the non-amphipathic alpha helix is from 20 to 30 amino acid residues in length.

90. The polypeptide of any one of embodiments 79-89, wherein the non-amphipathic alpha helix has a hydrophobic moment (pH) of less than 1.0.

91. The polypeptide of any one of embodiments 79-90, wherein the non-amphipathic alpha helix comprises an amino acid sequence of KARKKKLNKKGRKMAGRKRGR (SEQ ID NO: 11) or a variant thereof comprising an amino acid sequence that differs from KARKKKLNKKGRKMAGRKRGR (SEQ ID NO: 11) by no more than 6 amino acids.

92. The polypeptide of embodiment 91, wherein the non-amphipathic alpha helix comprises the amino acid sequence of KARKX₁KLX₂X₃KGRX₄MAGRKRGR (SEQ ID NO: 12), wherein X₁, X₂, X₃, and X₄ are each, independently, any amino acid.

93. The polypeptide of embodiment 92, wherein X₁, X₂, X₃, and X₄ are each, independently, selected from lysine, alanine, asparagine, arginine, and leucine.

94. The polypeptide of embodiment 93, wherein the non-amphipathic alpha helix comprises the amino acid sequence of KARKAKLRLKGRLMAGRKRGR (SEQ ID NO: 13).

95. The polypeptide of any one of embodiments 79-90, wherein the non-amphipathic alpha helix comprises an amino acid sequence having at least 85%, 90%, 95%, 97%, or 99% sequence identity to

| (SEQ ID NO: 11) | |
|---|---|
| | KARKKKLNKKGRKMAGRKRGR, |
| (SEQ ID NO: 14) | |
| | KARKKKLNKKGRKMAGRKRGRPK, |
| (SEQ ID NO: 15) | |
| | KARKAKLRLKGRLMAGRKRGRPK, |
| (SEQ ID NO: 16) | |
| | KARKAKLRLKGRLMAGRKRGRP, |
| (SEQ ID NO: 17) | |
| | KARKKKLNKKGRKMAGRKRGRP, |
| (SEQ ID NO: 18) | |
| | KARKAKLRLKARLWARHRARACEA, |
| | or |
| (SEQ ID NO: 19) | |
| | KARKAKLRLKGRLWARHRACEA. |

96. The polypeptide of embodiment 95, wherein the non-amphipathic alpha helix comprises the amino acid sequence of KARKKKLNKKGRKMAGRKRGR (SEQ ID NO: 11), KARKKKLNKKGRKMAGRKRGRPK (SEQ ID NO: 14), KARKAKLRLKGRLMAGRKRGRPK (SEQ ID NO: 15), KARKAKLRLKGRLMAGRKRGRP (SEQ ID NO: 16), KARKKKLNKKGRKMAGRKRGRP (SEQ ID NO: 17), KARKAKLRLKARLWARHRARACEA (SEQ ID NO: 18), or KARKAKLRLKGRLWARHRACEA (SEQ ID NO: 19).

97. The polypeptide of embodiment 95, wherein the polypeptide comprises an amino acid sequence having at least 85%, 90%, 95%, 97%, or 99% sequence identity to

| (SEQ ID NO: 20) | |
|---|---|
| | KARKKKLNKKGRKMAGRKRGRPKKKRKV, |
| (SEQ ID NO: 21) | |
| | KARKKKLNKKGRKMAGRKRGRPKGGGSPAAKRVKLD, |
| (SEQ ID NO: 22) | |
| | KARKAKLRLKGRLMAGRKRGRPKKSGGVKRKKKP, |
| (SEQ ID NO: 23) | |
| | KARKAKLRLKGRLMAGRKRGRPKGGGSPAAKRVKLD, |
| (SEQ ID NO: 24) | |
| | KARKAKLRLKGRLMAGRKRGRPCGGGSPAAKRVKL, |
| (SEQ ID NO: 25) | |
| | KARKAKLRLKGRLMAGRKRGRPCGGSPAAKRVKL, |
| (SEQ ID NO: 26) | |
| | KARKKKLNKKGRKMAGRKRGRPCGGSPKKKRKV, |
| (SEQ ID NO: 27) | |
| | KARKAKLRLKGRLMAGRKRGRPCGGSPKKKRKV, |
| (SEQ ID NO: 28) | |
| | KARKAKLRLKARLWARHRARACEAPAAKRVKL, |
| (SEQ ID NO: 29) | |
| | KARKAKLRLKGRLWARHRACEAPAAKRVKL, |
| | or |
| (SEQ ID NO: 30) | |
| | KARKAKLRLKGRLWARHRACEAPKKKRKV. |

98. The polypeptide of embodiment 97, wherein the polypeptide comprises the amino acid sequence of KARKKKLNKKGRKMAGRKRGRPKKKRKV (SEQ ID NO: 20), KARKKKLNKKGRKMAGRKRGRPKGGGSPAAKRVKLD (SEQ ID NO: 21), KARKAKLRLKGRLMAGRKRGRPKKSGGVKRKKKP (SEQ ID NO: 22), KARKAKLRLKGRLMAGRKRGRPKGGGSPAAKRVKLD (SEQ ID NO: 23), KARKAKLRLKGRLMAGRKRGRPCGGGSPAAKRVKL (SEQ ID NO: 24), KARKAKLRLKGRLMAGRKRGRPCGGSPAAKRVKL (SEQ ID NO: 25), KARKKKLNKKGRKMAGRKRGRPCGGSPKKKRKV (SEQ ID NO: 26), KARKAKLRLKGRLMAGRKRGRPCGGSPKKKRKV (SEQ ID NO: 27), KARKAKLRLKARLWARHRARACEAPAAKRVKL (SEQ ID NO: 28), KARKAKLRLKGRLWARHRACEAPAAKRVKL (SEQ ID NO: 29), or KARKAKLRLKGRLWARHRACEAPKKKRKV (SEQ ID NO: 30).

99. The polypeptide of any one of embodiments 79-98, wherein the polypeptide is from 2 kDa to 5 kDa.

100. The polypeptide of any one of embodiments 79-99, wherein the polypeptide is from 20 to 50 amino acid residues in length.

101. The polypeptide of any one of embodiments 79-100, wherein 30-50% of the residues of the polypeptide are cationic.

102. A composition comprising the polypeptide of any one of embodiments 79-101 and a polynucleotide.

103. The composition of embodiment 102, wherein the polynucleotide is from about 500 nucleotides to about 20,000 nucleotides in length.

104. The composition of embodiment 102 or 103, wherein the polynucleotide encodes a protein.

105. The composition of any one of embodiments 102-104, wherein the polynucleotide is a non-viral polynucleotide.

106. The composition of any one of embodiments 102-105, wherein the polynucleotide is a closed circular polynucleotide.

107. The composition of any one of embodiments 102-106, wherein the polynucleotide is DNA.

108. The composition of embodiment 106 or 107, wherein the polynucleotide is closed circular supercoiled DNA.

109. The composition of any one of embodiments 102-108, wherein the polypeptide and the polynucleotide are present at a molar ratio of from about 100:1 to about 10,000,000:1.

110. The composition of embodiment 109, wherein the polypeptide and the polynucleotide are present at a molar ratio of from about 1,000:1 to about 10,000:1.

111. The composition of any one of embodiments 102-110, further comprising a lipid.

112. The composition of embodiment 111, wherein the composition comprises a mixture of lipids.

113. The composition of embodiment 111 or 112, wherein the lipid comprises DLin-MC3-DMA, 1,2-DOPE, cholesterol, DMG-PEG 2000, or a combination thereof.

114. The composition of embodiment 113, wherein the composition comprises a mixture of DLin-MC3-DMA, DOPE, cholesterol, and DMG-PEG 2000.

115. The composition of any one of embodiments 111-114, wherein the composition comprises a nanoparticle comprising the polypeptide, the polynucleotide, and the lipid.

116. The composition of any one of embodiments 111-115, wherein the composition comprises a plurality of the polypeptides, a plurality of the polynucleotides, and a plurality of the lipids.

117. The composition of embodiment 116, wherein the composition comprises a plurality of nanoparticles.

118. The composition of embodiment 117, wherein at least 90% of the nanoparticles in the composition have a diameter from about 10 nm to about 500 nm as measured by dynamic light scattering (DLS).

119. The composition of embodiment 118, wherein at least 90% of the nanoparticles in the composition have a diameter from about 10 nm to about 250 nm as measured by DLS.

120. A method of introducing a polynucleotide into a target cell comprising contacting the target cell with the composition of any one of embodiments 102-119.

121. The method of embodiment 120, wherein the target cell is a mammalian cell.

122. The method of embodiment 121, wherein the mammalian cell is a human cell.

123. The method of any one of embodiments 120-122, wherein the contacting is performed by in vivo administration of the composition to a subject comprising the target cell.

124. The method of any one of embodiments 120-122, wherein the contacting with the target cell is performed in vitro.

125. The method of embodiment 124, wherein the target cell is introduced into the subject following the in vitro contacting.

126. The method of any one of embodiments 120-125, wherein the polynucleotide is expressed by the target cell.

127. The method of embodiment 126, wherein the expression is measured by detecting a protein encoded by the polynucleotide.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A composition comprising:
(a) a polypeptide comprising [A]-[B], wherein:
[A] is a DNA condensing polypeptide; and
[B] is a nuclear localization sequence (NLS);
(b) a polynucleotide; and
(c) a lipid.

2. The composition of claim 1, wherein the polypeptide is from 2 kDa to 5 kDa.

3. The composition of claim 1, wherein the polypeptide is from 20 to 50 amino acid residues in length.

4. The composition of claim 1, wherein 30-50% of the residues of the polypeptide are cationic.

5. The composition of claim 1, wherein the NLS is seven, eight, or nine amino acid residues in length.

6. The composition of claim 1, wherein the NLS comprises the amino acid sequence PKKKRKV (SEQ ID NO: 1).

7. The composition of claim 1, wherein the NLS comprises the amino acid sequence PAAKRVKL (SEQ ID NO: 2).

8. The composition of claim 1, wherein the NLS comprises the amino acid sequence of PAAKRVKLD (SEQ ID NO: 3) or VKRKKKP (SEQ ID NO: 4).

9. The composition of claim 1, wherein the polypeptide comprises a linker between [A] and [B].

10. The composition of claim 9, wherein the linker is from two to 20 amino acid residues in length.

11. The composition of claim 10, wherein the linker is from two to eight amino acid residues in length.

12. The composition of claim 11, wherein the linker is four or five amino acid residues in length.

13. The composition of claim 9, wherein the linker comprises one or more glycines or serines.

14. The composition of claim 9, wherein the linker comprises the amino acid sequence of GGGS (SEQ ID NO: 5), KSGG (SEQ ID NO: 6), CGGGS (SEQ ID NO: 7), or CGGS, (SEQ ID NO: 8).

15. The composition of claim 9, wherein the linker is a rigid linker.

16. The composition of claim 1, wherein the DNA condensing polypeptide comprises an amphipathic alpha helix.

17. The composition of any one of claim 16, wherein the amphipathic alpha helix is from 12 to 42 amino acid residues in length.

18. The composition of claim 17, wherein the amphipathic alpha helix is from 20 to 30 amino acid residues in length.

19. The composition of claim 16, wherein the amphipathic alpha helix comprises a RALA motif.

20. The composition of claim 19, wherein the amphipathic alpha helix comprises a plurality of RALA motifs.

21. The composition of claim 20, wherein the amphipathic alpha helix comprises three RALA motifs.

22. The composition of claim 1, wherein the DNA condensing polypeptide comprises an amino acid sequence having at least 85%, 90%, 95%, 97%, or 99% sequence identity to

| (SEQ ID NO: 9) | |
|---|---|
| | WEARLARALARALARHLARALARALRACEA. |

23. The composition of claim 22, wherein the DNA condensing polypeptide comprises the amino acid sequence of WEARLARALARALARHLARALARALRACEA (SEQ ID NO: 9).

24. The composition of claim 22, wherein the polypeptide comprises an amino acid sequence having at least 85%, 90%, 95%, 97%, or 99% sequence identity to

| (SEQ ID NO: 10) | |
|---|---|
| | WEARLARALARALARHLARALARALRACEAPKKKRKV. |

25. The composition of claim 24, wherein the polypeptide comprises the amino acid sequence of

| (SEQ ID NO: 10) | |
|---|---|
| | WEARLARALARALARHLARALARALRACEAPKKKRKV. |

26. The composition of claim 1, wherein the DNA condensing polypeptide comprises a DNA binding domain.

27. The composition of claim 26, wherein the DNA binding domain comprises an alpha helix.

28. The composition of claim 27, wherein the alpha helix is from 12 to 42 amino acid residues in length.

29. The composition of claim 28, wherein the alpha helix is from 20 to 30 amino acid residues in length.

30. The composition of claim 27, wherein the alpha helix is a non-amphipathic alpha helix.

31. The composition of claim 30, wherein the non-amphipathic alpha helix has a hydrophobic moment (pH) of less than 1.0.

32. The composition of claim 30, wherein the non-amphipathic alpha helix comprises an amino acid sequence of KARKKKLNKKGRKMAGRKRGR (SEQ ID NO: 11) or a variant thereof comprising an amino acid sequence that differs from KARKKKLNKKGRKMAGRKRGR (SEQ ID NO: 11) by no more than 6 amino acids.

33. The composition of claim 32, wherein the non-amphipathic alpha helix comprises the amino acid sequence of KARKX₁KLX₂X₃KGRX₄MAGRKRGR (SEQ ID NO: 12), wherein X₁, X₂, X₃, and X₄ are each, independently, any amino acid.

34. The composition of claim 33, wherein X₁, X₂, X₃, and X₄ are each, independently, selected from lysine, alanine, asparagine, arginine, and leucine.

35. The composition of claim 34, wherein the non-amphipathic alpha helix comprises the amino acid sequence of KARKAKLRLKGRLMAGRKRGR (SEQ ID NO: 13).

36. The composition of claim 30, wherein the non-amphipathic alpha helix comprises an amino acid sequence having at least 85%, 90%, 95%, 97%, or 99% sequence identity to

| (SEQ ID NO: 11) | |
|---|---|
| | KARKKKLNKKGRKMAGRKRGR, |
| (SEQ ID NO: 14) | |
| | KARKKKLNKKGRKMAGRKRGRPK, |
| (SEQ ID NO: 15) | |
| | KARKAKLRLKGRLMAGRKRGRPK, |
| (SEQ ID NO: 16) | |
| | KARKAKLRLKGRLMAGRKRGRP, |
| (SEQ ID NO: 17) | |
| | KARKKKLNKKGRKMAGRKRGRP, |
| (SEQ ID NO: 18) | |
| | KARKAKLRLKARLWARHRARACEA, |
| | or |
| (SEQ ID NO: 19) | |
| | KARKAKLRLKGRLWARHRACEA. |

37. The composition of claim 36, wherein the non-amphipathic alpha helix comprises the amino acid sequence of KARKKKLNKKGRKMAGRKRGR (SEQ ID NO: 11), KARKKKLNKKGRKMAGRKRGRPK (SEQ ID NO: 14), KARKAKLRLKGRLMAGRKRGRPK (SEQ ID NO: 15), KARKAKLRLKGRLMAGRKRGRP (SEQ ID NO: 16), KARKKKLNKKGRKMAGRKRGRP (SEQ ID NO: 17), KARKAKLRLKARLWARHRARACEA (SEQ ID NO: 18), or KARKAKLRLKGRLWARHRACEA (SEQ ID NO: 19).

38. The composition of claim 36, wherein the polypeptide comprises an amino acid sequence having at least 85%, 90%, 95%, 97%, or 99% sequence identity to KARKKKLNKKGRKMAGRKRGRPKKKRKV (SEQ ID NO: 20), KARKKKLNKKGRKMAGRKRGRPKGGGSPAAKRVKLD (SEQ ID NO: 21), KARKAKLRLKGRLMAGRKRGRPKKSGGVKRKKKP (SEQ ID NO: 22), KARKAKLRLKGRLMAGRKRGRPKGGGSPAAKRVKLD (SEQ ID NO: 23), KARKAKLRLKGRLMAGRKRGRPCGGGSPAAKRVKL (SEQ ID NO: 24), KARKAKLRLKGRLMAGRKRGRPCGGSPAAKRVKL (SEQ ID NO: 25), KARKKKLNKKGRKMAGRKRGRPCGGSPKKKRKV (SEQ ID NO: 26), KARKAKLRLKGRLMAGRKRGRPCGGSPKKKRKV (SEQ ID NO: 27), KARKAKLRLKARLWARHRARACEAPAAKRVKL (SEQ ID NO: 28), KARKAKLRLKGRLWARHRACEAPAAKRVKL (SEQ ID NO: 29), or KARKAKLRLKGRLWARHRACEAPKKKRKV (SEQ ID NO: 30).

39. The composition of claim 38, wherein the polypeptide comprises the amino acid sequence of

| (SEQ ID NO: 20) | |
|---|---|
| | KARKKKLNKKGRKMAGRKRGRPKKKRKV, |
| (SEQ ID NO: 21) | |
| | KARKKKLNKKGRKMAGRKRGRPKGGGSPAAKRVKLD, |
| (SEQ ID NO: 22) | |
| | KARKAKLRLKGRLMAGRKRGRPKKSGGVKRKKKP, |
| (SEQ ID NO: 23) | |
| | KARKAKLRLKGRLMAGRKRGRPKGGGSPAAKRVKLD, |
| (SEQ ID NO: 24) | |
| | KARKAKLRLKGRLMAGRKRGRPCGGGSPAAKRVKL, |
| (SEQ ID NO: 25) | |
| | KARKAKLRLKGRLMAGRKRGRPCGGSPAAKRVKL, |
| (SEQ ID NO: 26) | |
| | KARKKKLNKKGRKMAGRKRGRPCGGSPKKKRKV, |
| (SEQ ID NO: 27) | |
| | KARKAKLRLKGRLMAGRKRGRPCGGSPKKKRKV, |
| (SEQ ID NO: 28) | |
| | KARKAKLRLKARLWARHRARACEAPAAKRVKL, |
| (SEQ ID NO: 29) | |
| | KARKAKLRLKGRLWARHRACEAPAAKRVKL, |
| | or |
| (SEQ ID NO: 30) | |
| | KARKAKLRLKGRLWARHRACEAPKKKRKV. |

40. The composition of claim 1, wherein the polynucleotide is from about 500 nucleotides to about 20,000 nucleotides in length.

41. The composition of claim 1, wherein the polynucleotide encodes a protein.

42. The composition of claim 1, wherein the polynucleotide is a non-viral polynucleotide.

43. The composition of claim 1, wherein the polynucleotide is a closed circular polynucleotide.

44. The composition of claim 1, wherein the polynucleotide is DNA.

45. The composition of claim 43, wherein the polynucleotide is closed circular supercoiled DNA.

46. The composition of claim 1, wherein the polypeptide and the polynucleotide are present at a molar ratio of from about 100:1 to about 10,000,000:1.

47. The composition of claim 46, wherein the polypeptide and the polynucleotide are present at a molar ratio of from about 1,000:1 to about 10,000:1.

48. The composition of claim 1, wherein the lipid is a phospholipid.

49. The composition of claim 1, wherein the lipid is a cationic lipid.

50. The composition of claim 49, wherein the cationic lipid is 4-(dimethylamino)-butanoic acid, (10Z,13Z)-1-(9Z,12Z)-9,12-octadecadien-1-yl-10,13-nonadecadien-1-yl ester (DLin-MC3-DMA), 1,2-dioleoyl-3-dimethylammonium-propane (DODAP), 1,2-dioleyloxy-3-dimethylaminopropane (DODMA), N,N-dimethyl-2,2-di-(9Z,12Z)-9,12-octadecadien-1-yl-1,3-dioxolane-4-ethanamine (DLin-KC2-DMA), 4-(dimethylamino)-butanoic acid, (10Z,13Z)-1-(9Z,12Z)-9,12-octadecadien-1-yl-10,13-nonadecadien-1-yl ester (DLin-MC3-DMA), OF-02, 3,6-bis({4-[bis(2-hydroxydodecyl)amino]butyl})piperazine-2,5-dione (CKK-E12), 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), or 2-(dioctylamino)ethyl nonyl hydrogen phosphate (9A1P9).

51. The composition of claim 1, wherein the lipid is a PEGylated lipid.

52. The composition of claim 51, wherein the PEGylated lipid is 1,2-Dimyristoyl-sn-glycero-3-methoxypolyethylene glycol (DMG-PEG).

53. The composition of claim 52, wherein the DMG-PEG is DMG-PEG 2000.

54. The composition of claim 1, wherein the lipid is an anionic or neutral lipid.

55. The composition of claim 49, wherein the neutral lipid is 1,2-Dioleoyl-sn-glycero-3-phosphoethanolamine (DOPE).

56. The composition of claim 1, wherein the lipid is a sterol.

57. The composition of claim 56, wherein the sterol is cholesterol or a derivative thereof.

58. The composition of claim 1, wherein the composition comprises a mixture of lipids.

59. The composition of claim 1, wherein the composition comprises DLin-MC3-DMA, DOPE, cholesterol, DMG-PEG 2000, or a combination thereof.

60. The composition of claim 59, wherein the composition comprises a mixture of DLin-MC3-DMA, DOPE, cholesterol, and DMG-PEG 2000.

61. The composition of claim 60, wherein the mixture comprises DLin-MC3-DMA, DOPE, cholesterol, and DMG-PEG 2000 at a molar ratio of about 40-60:15-20:25-35:1-2.

62. The composition of claim 61, wherein the mixture comprises DLin-MC3-DMA, DOPE, cholesterol, and DMG-PEG 2000 at a molar ratio of about 50:18.5:30:1.5.

63. The composition of claim 1, wherein the composition comprises the polypeptide and the polynucleotide at a molar ratio of from about 1:1 to about 10,000:1.

64. The composition of claim 63, wherein the composition comprises the polypeptide and the polynucleotide at a molar ratio of from about 1000:1 to about 7,000:1.

65. The composition of claim 64, wherein the composition comprises the polypeptide and the polynucleotide at a molar ratio of about 1500:1, about 3000:1, or about 6000:1.

66. The composition of claim 1, wherein the composition comprises a nanoparticle comprising the polypeptide, the polynucleotide, and the lipid.

67. The composition of claim 1, wherein the composition comprises a plurality of the polypeptides, a plurality of the polynucleotides, and a plurality of the lipids.

68. The composition of claim 67, wherein the comprises a plurality of nanoparticles.

69. The composition of claim 68, wherein at least 90% of the nanoparticles in the composition have a diameter from about 10 nm to about 500 nm as measured by dynamic light scattering (DLS).

70. The composition of claim 69, wherein at least 90% of the nanoparticles in the composition have a diameter from about 10 nm to about 250 nm as measured by DLS.

71. A method of introducing a polynucleotide into a target cell comprising contacting the target cell with the composition of claim 1.

72. The method of claim 71, wherein the target cell is a mammalian cell.

73. The method of claim 72, wherein the mammalian cell is a human cell.

74. The method of claim 71, wherein the contacting is performed by in vivo administration of the composition to a subject comprising the target cell.

75. The method of claim 71, wherein the contacting with the target cell is performed in vitro.

76. The method of claim 75, wherein the target cell is introduced into the subject following the in vitro contacting.

77. The method of claim 71, wherein the polynucleotide is expressed by the target cell.

78. The method of claim 77, wherein the expression is measured by detecting a protein encoded by the polynucleotide.

79. A polypeptide comprising [C]-[L]-[D], wherein:
[C] is a non-amphipathic alpha helix from 12 to 42 amino acid residues in length;
[L] is a linker from one to 20 amino acid residues in length or is absent; and
[D] is a nuclear localization sequence (NLS) from five to 12 amino acid residues in length.

80. The polypeptide of claim 79, wherein the linker is from two to eight amino acid residues in length.

81. The polypeptide of claim 80, wherein the linker is four or five amino acid residues in length.

82. The polypeptide of claim 79, wherein the linker comprises one or more glycines or serines.

83. The polypeptide of claim 79, wherein the linker comprises the amino acid sequence of GGGS (SEQ ID NO: 5), KSGG (SEQ ID NO: 6), CGGGS (SEQ ID NO: 7), or CGGS, (SEQ ID NO: 8).

84. The polypeptide of claim 79, wherein the linker is a rigid linker.

85. The polypeptide of claim 79, wherein the NLS is seven, eight, or nine amino acid residues in length.

86. The polypeptide of claim 79, wherein the NLS comprises the amino acid sequence PKKKRKV (SEQ ID NO: 1).

87. The polypeptide of claim 79, wherein the NLS the amino acid sequence PAAKRVKL (SEQ ID NO: 2).

88. The polypeptide of claim 79, wherein the NLS comprises the amino acid sequence of PAAKRVKLD (SEQ ID NO: 3) or VKRKKKP (SEQ ID NO: 4).

89. The polypeptide of claim 79, wherein the non-amphipathic alpha helix is from 20 to 30 amino acid residues in length.

90. The polypeptide of claim 79, wherein the non-amphipathic alpha helix has a hydrophobic moment (pH) of less than 1.0.

91. The polypeptide of claim 79, wherein the non-amphipathic alpha helix comprises an amino acid sequence of KARKKKLNKKGRKMAGRKRGR (SEQ ID NO: 11) or a variant thereof comprising an amino acid sequence that differs from KARKKKLNKKGRKMAGRKRGR (SEQ ID NO: 11) by no more than 6 amino acids.

92. The polypeptide of claim 91, wherein the non-amphipathic alpha helix comprises the amino acid sequence of KARKX₁KLX₂X₃KGRX₄MAGRKRGR (SEQ ID NO: 12), wherein X₁, X₂, X₃, and X₄ are each, independently, any amino acid.

93. The polypeptide of claim 92, wherein X₁, X₂, X₃, and X₄ are each, independently, selected from lysine, alanine, asparagine, arginine, and leucine.

94. The polypeptide of claim 93, wherein the non-amphipathic alpha helix comprises the amino acid sequence of KARKAKLRLKGRLMAGRKRGR (SEQ ID NO: 13).

95. The polypeptide of claim 79, wherein the non-amphipathic alpha helix comprises an amino acid sequence having at least 85%, 90%, 95%, 97%, or 99% sequence identity to

| (SEQ ID NO: 11) | |
|---|---|
| | KARKKKLNKKGRKMAGRKRGR, |
| (SEQ ID NO: 14) | |
| | KARKKKLNKKGRKMAGRKRGRPK, |
| (SEQ ID NO: 15) | |
| | KARKAKLRLKGRLMAGRKRGRPK, |
| (SEQ ID NO: 16) | |
| | KARKAKLRLKGRLMAGRKRGRP, |
| (SEQ ID NO: 17) | |
| | KARKKKLNKKGRKMAGRKRGRP, |
| (SEQ ID NO: 18) | |
| | KARKAKLRLKARLWARHRARACEA, |
| | or |
| (SEQ ID NO: 19) | |
| | KARKAKLRLKGRLWARHRACEA. |

96. The polypeptide of claim 95, wherein the non-amphipathic alpha helix comprises the amino acid sequence of KARKKKLNKKGRKMAGRKRGR (SEQ ID NO: 11), KARKKKLNKKGRKMAGRKRGRPK (SEQ ID NO: 14), KARKAKLRLKGRLMAGRKRGRPK (SEQ ID NO: 15), KARKAKLRLKGRLMAGRKRGRP (SEQ ID NO: 16), KARKKKLNKKGRKMAGRKRGRP (SEQ ID NO: 17), KARKAKLRLKARLWARHRARACEA (SEQ ID NO: 18), or KARKAKLRLKGRLWARHRACEA (SEQ ID NO: 19).

97. The polypeptide of claim 95, wherein the polypeptide comprises an amino acid sequence having at least 85%, 90%, 95%, 97%, or 99% sequence identity to KARKKKLNKKGRKMAGRKRGRPKKKRKV (SEQ ID NO: 20), KARKKKLNKKGRKMAGRKRGRPKGGGSPAAKRVKLD (SEQ ID NO: 21), KARKAKLRLKGRLMAGRKRGRPKKSGGVKRKKKP (SEQ ID NO: 22), KARKAKLRLKGRLMAGRKRGRPKGGGSPAAKRVKLD (SEQ ID NO: 23), KARKAKLRLKGRLMAGRKRGRPCGGGSPAAKRVKL (SEQ ID NO: 24), KARKAKLRLKGRLMAGRKRGRPCGGSPAAKRVKL (SEQ ID NO: 25), KARKKKLNKKGRKMAGRKRGRPCGGSPKKKRKV (SEQ ID NO: 26), KARKAKLRLKGRLMAGRKRGRPCGGSPKKKRKV (SEQ ID NO: 27), KARKAKLRLKARLWARHRARACEAPAAKRVKL (SEQ ID NO: 28), KARKAKLRLKGRLWARHRACEAPAAKRVKL (SEQ ID NO: 29), or KARKAKLRLKGRLWARHRACEAPKKKRKV (SEQ ID NO: 30).

98. The polypeptide of claim 97, wherein the polypeptide comprises the amino acid sequence of

| (SEQ ID NO: 20) | |
|---|---|
| | KARKKKLNKKGRKMAGRKRGRPKKKRKV, |
| (SEQ ID NO: 21) | |
| | KARKKKLNKKGRKMAGRKRGRPKGGGSPAAKRVKLD, |
| (SEQ ID NO: 22) | |
| | KARKAKLRLKGRLMAGRKRGRPKKSGGVKRKKKP, |
| (SEQ ID NO: 23) | |
| | KARKAKLRLKGRLMAGRKRGRPKGGGSPAAKRVKLD, |
| (SEQ ID NO: 24) | |
| | KARKAKLRLKGRLMAGRKRGRPCGGGSPAAKRVKL, |
| (SEQ ID NO: 25) | |
| | KARKAKLRLKGRLMAGRKRGRPCGGSPAAKRVKL, |
| (SEQ ID NO: 26) | |
| | KARKKKLNKKGRKMAGRKRGRPCGGSPKKKRKV, |
| (SEQ ID NO: 27) | |
| | KARKAKLRLKGRLMAGRKRGRPCGGSPKKKRKV, |
| (SEQ ID NO: 28) | |
| | KARKAKLRLKARLWARHRARACEAPAAKRVKL, |
| (SEQ ID NO: 29) | |
| | KARKAKLRLKGRLWARHRACEAPAAKRVKL, |
| | or |
| (SEQ ID NO: 30) | |
| | KARKAKLRLKGRLWARHRACEAPKKKRKV. |

99. The polypeptide of claim 79, wherein the polypeptide is from 2 kDa to 5 kDa.

100. The polypeptide of claim 79, wherein the polypeptide is from 20 to 50 amino acid residues in length.

101. The polypeptide of claim 79, wherein 30-50% of the residues of the polypeptide are cationic.

102. A composition comprising the polypeptide of claim 79 and a polynucleotide.

103. The composition of claim 102, wherein the polynucleotide is from about 500 nucleotides to about 20,000 nucleotides in length.

104. The composition of claim 102, wherein the polynucleotide encodes a protein.

105. The composition of claim 102, wherein the polynucleotide is a non-viral polynucleotide.

106. The composition of claim 102, wherein the polynucleotide is a closed circular polynucleotide.

107. The composition of claim 102, wherein the polynucleotide is DNA.

108. The composition of claim 106, wherein the polynucleotide is closed circular supercoiled DNA.

109. The composition of claim 102, wherein the polypeptide and the polynucleotide are present at a molar ratio of from about 100:1 to about 10,000,000:1.

110. The composition of claim 109, wherein the polypeptide and the polynucleotide are present at a molar ratio of from about 1,000:1 to about 10,000:1.

111. The composition of claim 102, further comprising a lipid.

112. The composition of claim 111, wherein the composition comprises a mixture of lipids.

113. The composition of claim 111, wherein the lipid comprises DLin-MC3-DMA, 1,2-DOPE, cholesterol, DMG-PEG 2000, or a combination thereof.

114. The composition of claim 113, wherein the composition comprises a mixture of DLin-MC3-DMA, DOPE, cholesterol, and DMG-PEG 2000.

115. The composition of claim 111, wherein the composition comprises a nanoparticle comprising the polypeptide, the polynucleotide, and the lipid.

116. The composition of claim 111, wherein the composition comprises a plurality of the polypeptides, a plurality of the polynucleotides, and a plurality of the lipids.

117. The composition of claim 116, wherein the composition comprises a plurality of nanoparticles.

118. The composition of claim 117, wherein at least 90% of the nanoparticles in the composition have a diameter from about 10 nm to about 500 nm as measured by dynamic light scattering (DLS).

119. The composition of claim 118, wherein at least 90% of the nanoparticles in the composition have a diameter from about 10 nm to about 250 nm as measured by DLS.

120. A method of introducing a polynucleotide into a target cell comprising contacting the target cell with the composition of claim 102.

121. The method of claim 120, wherein the target cell is a mammalian cell.

122. The method of claim 121, wherein the mammalian cell is a human cell.

123. The method of claim 120, wherein the contacting is performed by in vivo administration of the composition to a subject comprising the target cell.

124. The method of claim 120, wherein the contacting with the target cell is performed in vitro.

125. The method of claim 124, wherein the target cell is introduced into the subject following the in vitro contacting.

126. The method of claim 120, wherein the polynucleotide is expressed by the target cell.

127. The method of claim 126, wherein the expression is measured by detecting a protein encoded by the polynucleotide.
